(19) United States
(12) Patent Application Publication
(10) Pub. No.: US 2025/0333771 A1
(43) Pub. Date: Oct. 30, 2025

(54) PROCESS FOR PRODUCING L-CYSTEIC ACID AND USE THEREOF

(71) Applicant: WACKER CHEMIE AG, Munich (DE)

(72) Inventors: Rupert Pfaller, Munich (DE); Günter WICH, Munich (DE)

(73) Assignee: WACKER CHEMIE AG, Munich (DE)

(21) Appl. No.: 18/864,602

(22) PCT Filed: May 31, 2022
(86) PCT No.: PCT/EP2022/064803

§ 371 Date: Nov. 11, 2024

(51) Int. Cl. C12P 13/12; C12N 9/10; C12N 9/16; C12N 9/88; C12P 13/00

(52) U.S. Cl. CPC C12P 13/12; C12N 9/1085; C12N 9/16; C12N 9/88; C12P 13/005; C12Y 205/01076; C12Y 301/03003; C12Y 401/01029

(57) ABSTRACT

Processes for producing L-cysteic acid along with uses for the same. The process includes providing a reaction of O-phospho-L-serine (OPS) with a salt of sulfurous acid (sulfite) and a cysteate synthase (CS enzyme) belonging to enzyme class EC 2.5.1.76 in a biotransformation. Where a concentration of OPS in a batch is at least 1 g/L and the CS enzyme is produced in an enzymatically active form by growth of the microorganism strain E. coli K12 JM105. The biotransformation is carried out under active pH control and the CS enzyme has an amino acid sequence specified in SEQ ID NO: 4 or an amino acid sequence homologous thereto. Where an amino acid sequence homologous to SEQ ID NO: 4 has a sequence identity of at least 50% in relation to SEQ ID NO: 4 and, at the same time, cysteate synthase enzyme activity.

The invention relates to a process for producing L-cysteic acid, comprising the reaction of O-phospho-L-serine (OPS) with a salt of sulfurous acid (sulfite) and a cysteate synthase (CS enzyme) belonging to enzyme class EC 2.5.1.76 in a biotransformation. The L-cysteic acid produced according to the invention can be decarboxylated to taurine. The invention further relates to the use of the L-cysteic acid produced for production of taurine.

L-cysteic acid ((R)-2-amino-3-sulfopropanoic acid, 3-sulfo-L-alanine, CAS 498-40-8) is a non-proteinogenic L-amino acid that can be detected in nature as an oxidation product of the proteinogenic amino acid L-cysteine, for example in sheep's wool. Cysteic acid is also an intermediate of coenzyme M biosynthesis (CoM, 2-mercaptoethanesulfonic acid, CAS 3375-50-6) by methanogenic archaebacteria. The biosynthesis pathway of L-cysteic acid in methanogenic bacteria proceeds from 0-phospho-L-serine (OPS, L-serine-O-phosphate, L-2-amino-3-hydroxypropanoic acid 3-phosphate, CAS 407-41-0), which reacts with sulfite in a reaction catalyzed by a cysteate synthase (CS enzyme) to form L-cysteic acid, according to equation (1):

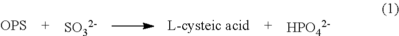

Graham et al., Biochem. J. (2009) 424: 467-478 disclose that, in the case of a gene from Methanoscarcina acetivorans that is related to threonine synthases and that is produced recombinantly in E. coli and enriched as enzymatically inactive protein in so-called inclusion bodies, detectable enzyme activity is only measurable after complicated renaturation of the inclusion bodies. Following renaturation, it was possible to demonstrate the production of L-cysteic acid from the reaction of commercially available (chemically synthesized) OPS and sulfite according to equation (1) in analytical assays (HPLC and mass spectrometry) with the renatured protein. As a result of these investigations, the renatured protein from M. acetivorans was associated with the activity of a cysteate synthase and its genetic relation to threonine synthases was established.

L-Cysteic acid can be produced chemically, for example by oxidation of cysteine with chlorine in an alcoholic solution, with bromine in HCl or iodine HCl in DMSO, or by oxidative cleavage of cystine. Furthermore, L-cysteic acid can also be produced by oxidation of L-cysteine sulfinic acid. The known processes for chemical production of L-cysteic acid, which are not considered sustainable, use environmentally hazardous chemicals and have low consumer acceptance, especially in applications in the food, cosmetics and pharmaceutical sectors.

L-cysteic acid can be used, for example, in fish farming or in the cosmetics sector, for example as an ingredient of Regu®-Slim (DSM) for skin care. In peptide chemistry, L-cysteic acid is used as a water-soluble protective group. Furthermore, L-cysteic acid can be converted to taurine by decarboxylation.

Graham et al. (2009, see above) showed that, when the CS enzyme having a molecular weight of 44 kDa is heterologously expressed in E. coli, it is not produced in active form, but only enriched in inactive form in inclusion bodies. Solubilization of the inclusion bodies and refolding to form the active enzyme led to a very low yield of 3 mg of refolded protein per L of E. coli culture and was thus unsuited to a preparative biotransformation process for producing L-cysteic acid on an industrial scale.

According to Graham et al. (2009, see above), enzyme activity in the absence of refolding was only detectable when the CS enzyme was expressed as a 103 kDa CS fusion protein, though both enzyme yield and a specific enzyme activity of 0.015 U/mg protein were very low. This prior art shows that active CS enzyme is only producible in E. coli with great effort in a costly process and with low yields.

In a metabolic engineering approach, Joo et al. (2018), J. Agric. Food Chem. 66: 13454-13463, describe a genetically modified and sulfur utilization-optimized strain of the bacterium Corynebacterium glutamicum for the production of taurine that used the CS gene from M. acetivorans. For the gene expressed in C. glutamicum, CS activity was analytically detected by enzyme assays. The activity of the CS enzyme in taurine-producing strains was indirectly inferred. As shown in Fig. S2 of the “Supporting Information” in Joo et al. (2018, see above), CS-expressing C. glutamicum cells mainly intracellularly accumulate OPS, the substrate of the CS reaction, and additionally serine, cysteine and taurine. Details about the production of L-cysteic acid were not given. Joo et al. (2018, see above) show that the simultaneous presence of OPS and CS enzyme in a cell is insufficient for the effective production of L-cysteic acid, even if the intracellular production of other sulfur-containing compounds such as cysteine and taurine takes place in the same strain with measurable yields.

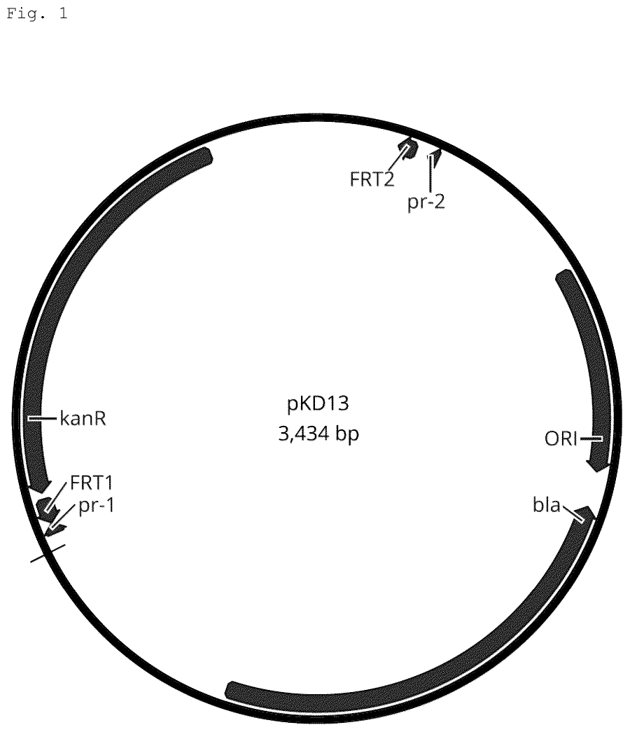

Tevatia et al., Algal Research (2015) 9: 21-26, describe the natural production of taurine in microalgae, with L-cysteic acid also being detected as an intermediate. As described in Fig. 1 of Tevatia et al. (2015, see above), a biosynthesis pathway in algae leads from L-serine to L-cysteic acid (“Cysteate” in Fig. 1). However, the synthesis pathway does not contain the enzymatic reaction of a CS enzyme according to equation (1). None of the biosynthesis pathways described leads to L-cysteic acid via OPS. The intracellular content of L-cysteic acid was very low and was accompanied by multiple by-products that make workup more difficult, such as methionine, cysteine, cysteine sulfinic acid, hypotaurine and taurine, and so the growth of microalgae is unsuitable for the production of L-cysteic acid.

In a metabolic engineering approach, US 2019/0062757A1 (KnipBio) describes heterologous production strains for the production of taurine or its precursors. Described were strains expressing the CS enzyme and other biosynthetic genes from taurine metabolism in various configurations. The yields achieved for hypotaurine and taurine were very low with a maximum of 419 ng/ml. Yields for the production of L-cysteic acid were not mentioned. In this prior art, CS gene constructs were used, but an enzyme assay to detect CS enzyme activity was not carried out. Furthermore, it was not shown that L-cysteic acid is biotechnologically producible in preparative amounts by this metabolic engineering approach. Likewise, it was not disclosed whether the strains for the production of OPS or CS enzyme described in the document are suitable for use in a biotransformation to produce L-cysteic acid according to equation (1).

Steinfeld et al., ACS Chem. Biol. (2014) 9: 1104-1112 describe the increased intracellular production of OPS in an E. coli strain having a deleted serB gene. Increased extracellular production of OPS was not described (see Fig. 5 in Steinfeld et al.).

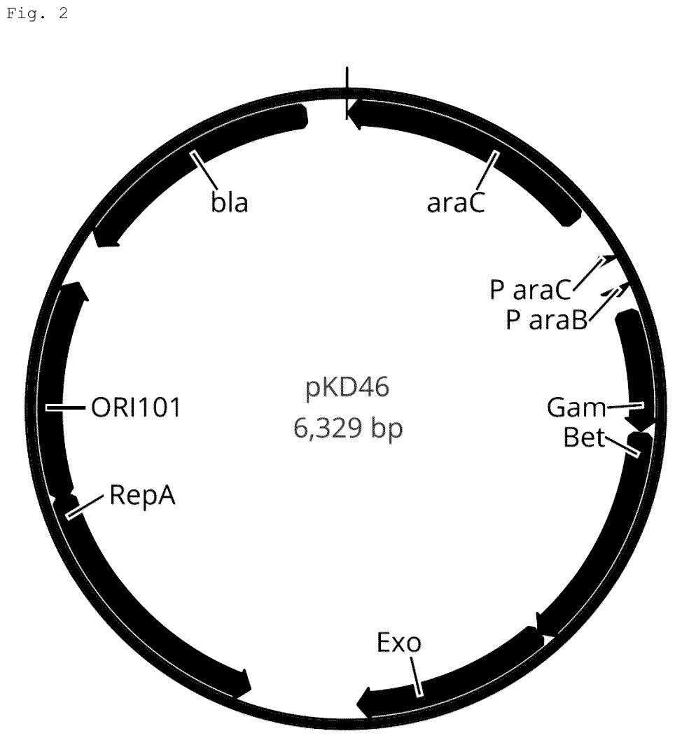

EP 2 444 481 B1 from CJ CheilJedang Corporation (KR) describes a process for producing L-cysteine, using a strain having reduced SerB activity to produce OPS, which is then reacted in an enzymatically catalyzed reaction with sulfide or thiosulfate to form L-cysteine. It is not known that the enzyme class of the 0-phosphoserine sulfhydrylases used here (OPSS, EC 2.5.1.65) can also utilize sulfite as substrate in the sense of a CS enzyme. In fact, Steiner et al. J. Bacteriol. (2014), 196: 3410-3420 investigate the reaction mechanism of the OPSS enzyme from Mycobacterium tuberculosis and find that, in contrast to Na₂S (sulfide) and Na₂S₂O₃ (thiosulfate), salts of sulfurous acid, such as Na₂S₂O₅ and Na₂SO₃, are unsuitable as S-donors for the reaction with an enzyme-bound aminoacrylate intermediate stemming from the binding of OPS to the OPSS enzyme with elimination of phosphate (see Fig. 2 in Steiner et al., 2014, see above). EP 2 444 481 B1 thus discloses the inactivation of the serB gene, as also known from Steinfeld et al. (2014, see above), to produce strains which then produce OPS. The OPS was then used in a biotransformation with OPSS enzymes to produce cysteine.

The biotechnological production of OPS is known in the prior art. However, the prior art does not provide a method of production for the CS enzyme that would be suitable for providing CS enzyme of sufficient activity for a hitherto unknown industrially applicable process of biotransformation of OPS with a sulfite, in which L-cysteic acid is produced on a preparative scale for further use.

There is therefore a need for an environmentally friendly and sustainable process for producing L-cysteic acid on a preparative scale, suited to which is a biotechnological process. Owing to the consumer-driven trend away from chemically produced ingredients, a goal of the invention is a biotechnological process for producing L-cysteic acid by biotransformation that is suitable for use on an industrial scale.

It is an object of the present invention to provide an industrially applicable process for cost-effective production of L-cysteic acid by biotransformation of OPS, instead of a chemical process and avoiding a L-cysteic acid production strain produced by metabolic engineering, and to use the L-cysteic acid thus produced in a further use, for example for production of taurine.

The object is achieved by a process for producing L-cysteic acid, comprising the reaction of O-phospho-L-serine (OPS) with a salt of sulfurous acid (sulfite) and a cysteate synthase (CS enzyme) belonging to enzyme class EC 2.5.1.76 in a biotransformation.

In the context of the present invention, production processes are distinguished as follows:
1. Chemical processes
2. Biotechnological processes:
   a) by metabolic engineering
      Metabolic engineering (also called “pathway design”), in contrast to biotransformation, is a biotechnology method in which metabolic pathways of an organism are modified by optimization or modification of genetic and regulatory processes. New or modified enzymes can be introduced into an organism by supplementation of the genome with genes of enzymes, or genes of endogenous enzymes can be expressed at an enhanced or attenuated level, thereby establishing new metabolic pathways in an organism or enhancing or attenuating existing metabolic pathways. The goal of metabolic engineering is for the organism to produce either a new metabolite or a cell-endogenous metabolite at increased yield. A metabolic engineering process does not use starting materials specific for the metabolite, such as an enzyme substrate, for example OPS in the present invention; instead, it only uses a nutrient medium which is required for the growth of the organism in question and is composed of a carbon source (e.g., glucose), a nitrogen source (e.g., an ammonium salt or a complex amino acid mixture such as peptone or yeast extract) and other salts required for growth. Such nutrient media are known to a person skilled in the art from microbiological practice.
   b) by biotransformation
      Biotransformation is defined as the transformation of one or more reactants into a product under enzymatic catalysis, the enzyme substrate being added with the enzyme to a reaction batch. In the reaction batch, the enzyme substrate added, such as OPS in the present invention, is converted enzymatically, in the present invention by an enzyme selected from the class of cysteate synthases (CS enzyme, EC 2.5.1.76) in the presence of a salt of sulfurous acid. The reactant(s) can stem from chemical or biotechnological production. The OPS used in the process according to the invention can stem, for example, from chemical synthesis or from biotechnological production by growth of a production strain. The enzyme used for the enzymatic catalysis preferably stems from biotechnological production by fermentation of a production strain from the Enterobacteriaceae family that heterologously expresses the CS enzyme.

An advantage of the present invention is that the process according to the invention for producing L-cysteic acid from OPS and sulfite with the aid of the CS enzyme is a biotransformation process. This means that it is a highly targeted, specific reaction and does not require, for example, complex steps of culturing or steps of purification from a microorganism culture. Furthermore, through optimization of the reaction conditions and of the amount of reactants and enzyme used, biotransformation makes it possible to achieve high space-time yields in a simple manner, this being far more difficult by strain optimization in the case of metabolic engineering. Altogether, the process according to the invention is implementable and controllable in an economically simple manner.

The reaction (1) according to the invention is catalyzed by the enzyme cysteate synthase (CS enzyme) belonging to enzyme class EC 2.5.1.76. CS enzyme in enzymatically active form refers to a protein capable of catalyzing the synthesis of L-cysteic acid from OPS and a salt of sulfurous acid as described in the following CS enzyme activity assay.

The CS enzyme activity assay can be carried out as follows:
i) A CS enzyme produced by growth in a shake flask or in a fermentation can be used in the reaction as follows:
   as an aliquot from the culture broth without further workup or
   as an aliquot of the cell suspension after reisolation of the cells from the culture broth, for example by centrifugation, or
   in the form of an aliquot of the cell homogenate
   a) after mechanical disruption of the cell suspension or
   b) in the form of chemically permeabilized cells (e.g., by chloroform)
   or
   as a cell extract after removal of particulate constituents from the cell homogenate or
   as an enzyme purified by chromatography, for example.
   As described in example 3 of the present invention, the total protein concentration obtained in each case can be determined, for example, by means of a Qubit 3.0 Fluorometer from Thermo Fisher Scientific using the “Qubit© Protein Assay Kit” according to the manufacturer's instructions.
ii) A solution buffered to pH 7 with potassium phosphate is initially charged with OPS (10 mM final concentration) and Na sulfite (20 mM final concentration) and the reaction is started by addition of the CS enzyme. The assay volume is 10 ml. The temperature at which the assay is carried out is 30° C. The amount of CS enzyme used depends on the purity. If culture broth, cell suspension of reisolated cells or cell homogenate is used, at least 0.1 mg of the enzyme fractions prepared in i) is used. In the case of purified CS enzyme, at least 10 μg of the purified enzyme fraction are used. 1 h, 2 h and 4 h after the start of the reaction, 1 ml of the assay is removed in each case and centrifuged for 10 min and the content of OPS and L-cysteic acid is determined by calibrated HPLC (see example 4); the reference substances used for calibration are commercially available (Sigma-Aldrich).

The process according to the invention is preferably characterized in that the CS enzyme is produced by growth of a microorganism strain from the Enterobacteriaceae family, it being preferred that the cds encoding the CS enzyme is expressed heterologously and particularly preferably in enzymatically active form in the microorganism strain from the Enterobacteriaceae family.

Heterologous expression is understood to mean the expression of the cds of a gene or the cds of part of a gene in a host organism which by nature does not have said gene or gene fragment. Introducing the cds of the heterologous gene into the host organism encompasses the use of recombinant DNA technology. The cds of the heterologous gene can be introduced into the host organism by integration into the genome thereof or extrachromosomally in the form of an autonomously replicating gene construct (plasmid, vector).

Preference is given to introducing the cds of the heterologous gene into the host organism in the form of an autonomously replicating gene construct (plasmid, vector). A distinction is made between constitutive expression and induced expression, depending on the genetic element (promoter) used to control the expression of the cds of the heterologous gene. In the case of constitutive expression, gene expression is active during all phases of cell cultivation (unregulated). In the case of induced expression (regulated), gene expression is stimulated by addition of an inducer molecule to the cell culture, for example addition of the inducer molecule IPTG for induction of the tac promoter in examples 3 and 7 of the present invention. Preference is given to induced expression, in which gene expression is stimulated by addition of an inducer molecule to the cell culture.

Homologous expression by contrast involves the overexpression of the cds of a gene in a host organism from the genome of which said gene originally stems.

If a heterologously expressed protein is an enzyme for example, it may be in enzymatically active form or may be enriched in enzymatically inactive form in, for example, inclusion bodies.

Accordingly, heterologous expression of the CS enzyme in enzymatically active form means that i) the cds of the gene encoding the CS enzyme that is introduced into the host strain is not coded in the genome of the host strain, ii) at least the cds of the gene encoding the CS enzyme is integrated chromosomally in the genome of the host organism by recombinant DNA technology or is preferably introduced extrachromosomally into the host organism by an autonomously replicating vector, and iii) that a CS enzyme is expressed in enzymatically active form by said cds. Particularly preferably, the heterologously expressed CS enzyme is expressed in enzymatically active form by the microorganism strain. This means that the heterologously introduced cds expresses a CS enzyme which, following protein biosynthesis and any posttranslational modification, for example the incorporation of a cofactor such as pyridoxal phosphate in the case of the CS enzyme (see the entry in the KEGG enzyme database under the entry number EC2.5.1.76), is in enzymatically active form in the microorganism. “Expressed in enzymatically active form” does not mean that the protein is initially produced as an inactive protein in inclusion bodies and is in enzymatically active form only after renaturation.

In summary, the process in an especially preferred embodiment is characterized in that the CS enzyme is produced by growth of a microorganism strain from the Enterobacteriaceae family that expresses the CS enzyme heterologously and in enzymatically active form.

In the context of the present invention, a reaction batch is defined as a mixture of reactant (starting material), enzyme and optionally other reactants, in which the reactant is converted into a product.

The yield of the reaction within the meaning of the invention is defined as the amount of reactant used that is converted into the product under reaction conditions. The yield can be expressed as absolute yield of the product (mmol or g), as a volume yield in terms of absolute amount of product per unit volume (mM or g/L) or as a relative yield of product as a percentage of reactant used (taking into account the molecular weights of the reactant and of the product), also referred to as percent yield.

In the context of this invention, the term “growth” or, synonymously, “culture” of microorganism cells covers both shake-flask culture processes and fermentative processes. The medium used for growth or culture of microorganisms is referred to as growth medium or culture medium or, in the case of fermentation, also as fermentation medium. Growth/culture/fermentation of the production strain in the growth, culture or fermentation medium yields the culture broth/fermenter broth. The culture broth/fermenter broth consists of the biomass of the cells of the production strain and of the biomass-free culture supernatant/fermentation supernatant that formed over the course of growth from the growth medium and from the metabolites secreted by the cells.

Fermentation is a process step for the production (cultivation) of cell cultures on an industrial scale (production scale), in which a preferably microbial production strain is made to grow under defined conditions of culture medium, temperature, pH, oxygen supply, and mixing of the medium. Depending on the configuration (genetic makeup) of the production strain, the aim of fermentation is to produce a protein/enzyme or a metabolite, in each case in the highest possible yield for further use. The components of the process according to the invention, OPS and CS enzyme, can be produced by fermentation. The final product of fermentation is a fermenter broth consisting of the biomass of the cells of the production strain (fermenter cells) and of the biomass-free fermentation supernatant that formed over the course of fermentation from the growth medium and from the metabolites secreted by the fermenter cells. The target products of fermentation can be present in the fermenter cells or in the fermentation supernatant. For instance, OPS is found in the fermentation supernatant, whereas the CS enzyme is found in the fermenter cells.

Shake flask growth is used to cultivate microorganisms on a laboratory scale, in contrast to a production scale through fermentation. Although a shake flask culture also involves specifying a particular medium and a pH and cultivation in the presence of oxygen under constant motion (shaking), more defined conditions concerning the medium, temperature, pH, oxygen supply, and mixing of the medium can be established and regulated in a fermenter. A smaller-scale culture, for example in a shake flask, can also be used as a preculture for inoculation of a larger-scale culture, for example a fermenter.

A production strain is defined as a microorganism strain suitable for production of a product, for example by fermentation. The production strain is distinguished by the fact that it is capable of (improved) production of the product as a result of genetic modification. The genetic modification can be due to a modification of the genome (chromosomal modification), due to introduction an autonomously replicating extrachromosomal genetic element such as a plasmid and due to a combination of a chromosomal and an extrachromosomal modification. An example of a production strain having a chromosomal modification is the strain E. coli W3110-ΔserB described in example 1 relating to the production of OPS. An example of a production strain produced by introduction of a plasmid is the strain E. coli JM105×pCSma-pKKj described in example 3 relating to the production of the CS enzyme. The microorganism strain bearing the extrachromosomal genetic element is referred to as the host strain or host organism and the extrachromosomal genetic element is referred to as a gene construct, plasmid, vector or expression vector.

Open reading frame (ORF, synonymous with cds or coding sequence) refers to a region of DNA or RNA that begins with a start codon and ends with a stop codon and encodes the amino acid sequence of a protein. The ORF is also referred to as the coding region or structural gene.

Gene refers to the section of DNA that contains all the basic information for producing a biologically active RNA. A gene contains the section of DNA from which a single-stranded RNA copy is produced by transcription and also the expression signals involved in the regulation of this copying process. The expression signals include for example at least a promoter, a transcription start, a translation start, and a ribosome binding site (RBS). A terminator and one or more operators are additional possible expression signals.

An mRNA, also known as messenger RNA, is a single-stranded ribonucleic acid (RNA) that carries the genetic information for the synthesis of a protein. An mRNA provides the assembly instructions for a particular protein in a cell. The mRNA molecule conveys the requisite message for protein synthesis from the genetic information (DNA) to the ribosomes responsible for protein synthesis. In a cell it is formed as a transcript of a section of DNA corresponding to a gene. The genetic information stored in the DNA is unchanged by this process.

Genes of eukaryotic organisms are predominantly what are known as mosaic genes and, unlike prokaryotic genes, also contain noncoding sections known as introns (intragenic regions). Coding sequences, which are known as exons (expressed regions), are sections of DNA of a eukaryotic gene that, after being transcribed into RNA, are translated by the ribosomes into the amino acid sequence of a protein. After transcription of DNA into RNA, the introns are spliced from the primary transcript. The protein-coding RNA free of introns is termed messenger RNA (mRNA), or “mature” mRNA. This undergoes further modifications such as capping and polyadenylation. The coding region of the mature mRNA is then translated into the protein sequence. If a eukaryotic gene containing an exon/intron structure is to be expressed in prokaryotic organisms, it is necessary to back-translate the protein sequence or coding region of the mature mRNA into intron-free DNA, since no processing of the exon/intron structure takes place in prokaryotes. When referring in the context of this invention to gene sequences derived from the protein sequence or gene sequences derived from mRNA, what is meant is precisely this process of back-translation. It is preferable that sequence optimization, i.e. adaptation to the codon usage of the corresponding prokaryote (codon optimization), takes place concomitantly with the back-translation of the protein sequence or the mRNA sequence into a DNA sequence.

A gene construct refers to a DNA molecule in which a gene is linked to other genetic elements (e.g., promoter, terminator, selection marker, origin of replication). A gene construct in the context of the invention is a circular DNA molecule and is referred to as a plasmid, vector or expression vector. The genetic elements of the gene construct give rise to the extrachromosomal inheritance thereof during cell growth and to the production of the protein encoded by the gene.

The abbreviation WT (Wt) refers to the wild type. Wild-type gene refers to the form of the gene that arose naturally through evolution and is present in the wild-type genome. The DNA sequence of Wt genes is publicly accessible in databases such as the NCBI (National Center for Biotechnology Information) database. A microorganism strain having a Wt genome is referred to as Wt strain.

L-cysteic acid from the biotransformation of OPS with a salt of sulfurous acid, in accordance with the invention, can be either used further directly without further workup steps or enriched or purified by means of known methods. Such methods are known to a person skilled in the art from methods for isolating amino acids. Examples include filtration, centrifugation, extraction, adsorption, ion-exchange chromatography, precipitation, crystallization.

Preferably, the process is characterized in that the reaction batch containing the L-cysteic acid is used further without further workup, purification or isolation steps.

In an alternatively preferred embodiment, the process is characterized in that the L-cysteic acid produced is isolated from the reaction batch.

Denaturation in the context of this invention refers to a structural change in molecules such as proteins that is associated with a loss of biological function in the molecules, even though the primary structure thereof remains unchanged.

A denatured protein is characterized in that it is enzymatically inactive, i.e., in the case of CS enzymes in the context of the present invention, they are not capable of catalyzing the synthesis of L-cysteic acid from OPS and a salt of sulfurous acid. Denaturation can be due to physical or chemical influences. Incorrectly or incompletely folded, enzymatically inactive proteins can accumulate in the cell in protein aggregates (known as inclusion bodies) and can be regarded as naturally denatured proteins. Inclusion bodies are especially observed at a high level of expression, when the resultant high concentration of newly synthesized protein chains means that the aggregation thereof is favored over the folding thereof into the enzymatically active three-dimensional form. Whether a heterologously expressed protein will occur in the form of insoluble inclusion bodies or in active form cannot be predicted and depends not only on the primary structure of the protein chain (succession in the amino acid sequence), but also on the expression system and parameters used, by means of which the rate of protein biosynthesis can be controlled (e.g., growth temperature, strength of induction at inducible promotors). However, in the case of the present invention, it was surprising that the cysteate synthase from Methanoscarcina acetivorans was produced in enzymatically active form in E. coli, since according to the prior art (Graham et al., 2009, see above) the same protein was only produced in inactive form in inclusion bodies when heterologously expressed in E. coli.

Renaturation refers to the back-transformation of the denatured proteins into their biologically active spatial structure. Renaturation in protein biochemistry involves using chaotropic compounds to bring denatured protein in inclusion bodies back into solution, before the chaotropic compound is removed to allow protein renaturation. Urea and guanidine hydrochloride in particular are used in protein chemistry for this purpose.

Preferably, the process is characterized in that the CS enzyme is used in the reaction without a prior renaturation step.

Renaturation steps include the following process steps: dissolving the denatured protein in a medium containing a chaotropic compound and then removing the chaotropic compound. The extent to which the chaotropic compound has to be removed depends on the particular protein. The chaotropic compound can be removed, for example, by dialysis, selective binding of the chaotropic compound to a support material, selective binding of the protein to be renatured to a support material and subsequent elution under renaturing conditions, or dilution of the chaotropic compound below a critical concentration below which there is no longer a denaturing effect (see, for example, Graham et al., 2009, see above). Methods of protein renaturation have been described in the prior art. Renaturing conditions include, for example, dissolving the denatured protein in a 6 M aqueous urea solution or 6 M aqueous guanidine HCl solution.

Dilution or removal of the chaotropic compound below a critical concentration depends on the particular protein and means that the concentration of the chaotropic compound such as urea or guanidine is lowered below a concentration at which the protein in question can reassume the three-dimensional structure of its active form.

Chaotropic compound refers to chemical substances which disrupt ordered hydrogen bonds in water. Chaotropic compounds include barium salts such as barium chloride or barium acetate, guanidine hydrochloride, thiocyanates such as guanidinium thiocyanate, perchlorates, iodides, butanol, phenol, thiourea, urea and/or surfactants. Surfactants (also referred to as detergents or soaps) are organic compounds which act as surface-active substances, i.e., owing to their structure, they are arranged in the interface between two phases such that they lower the surface tension and as a result allow, for example, wetting. By lowering the surface tension, they further the commixing of two phases, possibly up to the formation of an emulsion. Surfactants are distinguished by a polarity of the molecular structure, with one part of the molecule having hydrophilic properties, which mediate solubility in water, and the other part of the molecule having hydrophobic properties, which allow surfactants to solubilize hydrophobic compounds and contribute to solubilization in water. The surfactants used are nonionic surfactants (polyalkylene glycol ethers, fatty alcohol propoxylates, alkyl glucosides, alkyl polyglucosides, octylphenol ethoxylates such as Triton X-100, nonylphenol ethoxylates), anionic surfactants (alkyl carboxylates, alkyl benzenesulfonates, alkyl sulfonates, fatty alcohol sulfates such as sodium lauryl sulfate, alkyl ether sulfates, sulfoacetates), cationic surfactants based on quaternary ammonium compounds (distearyldimethylammonium chloride, “Esterquat”) and/or zwitterionic (amphoteric) surfactants based on betaine (e.g., “cocoamidopropyl betaine”) or sulfobetaine (e.g., cocamidopropyl hydroxysultaine).

In a preferred embodiment, the process is characterized in that the CS enzyme is used in the reaction without a prior renaturation step. It is highly economically advantageous to work without a renaturation step because the complex, cost-intensive and environmentally harmful renaturation steps described above are not required. In the case of production of the CS enzyme by growth of a production strain, especially if the CS enzyme is produced by fermentation, it is moreover not necessary for the host cells expressing the CS enzyme to be first mechanically or chemically disrupted in a process which is complex and cost-intensive and is harmful to the environment, for example due to waste, and for inclusion bodies, in which for example the enzyme is enriched in inactive form in Graham et al. (2009), to be isolated from the cell lysate and renatured in order to be able to use the protein for enzymatic reactions.

The process according to the invention for producing L-cysteic acid by biotransformation requires the availability of OPS. OPS can be produced chemically or biotechnologically, for example by fermentation of an OPS production strain. Possible methods for chemical production of OPS are, for example, phosphorylation of L-serine, or the production of the racemate O-phospho-D/L-serine, which can be used directly, or OPS is obtained from the racemate beforehand, for example by resolution.

Preferably, the process for producing L-cysteic acid is characterized in that the OPS used in the reaction is produced biotechnologically. This is achieved by growth of an OPS production strain. Particular preference is given to the biotechnological production of OPS by growth of an OPS production strain in which OPS accumulates in the cell culture supernatant (extracellularly).

A person skilled in the art can use isotope analysis to determine whether a substance they wish to use as reactant in the process, such as OPS, stems from chemical or biotechnological, for example fermentative, production. An isotope analysis method capable of differentiating is described for example in Sieper et al., Rapid Commun. Mass Spectrom. (2006) 20: 2521-2527 and is based on determination of the isotope ratios for e.g. carbon or nitrogen, which vary according to whether a product stems from chemical (petroleum-based) production or biotechnological, for example fermentative (plant-based), production. A process for producing OPS is a biotechnological production process, for example fermentative (plant-based) production process, if, for example, the glucose used for growth of the production strain comes from plant-based production, which is also applicable to the process described in example 2.

In the cysteine metabolism of, for example, Escherichia coli, OPS serves as a biosynthetic precursor of L-serine. The latter is formed by dephosphorylation of OPS. This reaction is catalyzed enzymatically by O-phospho-L-serine phosphatases (SerB, EC 3.1.3.3). It is known from the prior art that E. coli strains having suppressed SerB activity can accumulate OPS. A microorganism strain having suppressed SerB activity is therefore characterized in that it is no longer able to produce L-serine by dephosphorylation of OPS and can consequently accumulate OPS.

Preferably, the process is characterized in that the OPS used in the reaction is produced using a microorganism strain having suppressed activity of the O-phospho-L-serine phosphatase (SerB enzyme) belonging to enzyme class EC 3.1.3.3. This means that, in this case, a microorganism strain having suppressed SerB activity is used as the OPS production strain, the suppression of SerB activity comprising a genetic modification in the microorganism strain. The SerB activity of the non-genetically modified microorganism strain (Wt strain) is set at 100%, and a microorganism strain having suppressed SerB activity is defined in that it contains lower SerB activity compared to the 100% activity in the Wt strain, preferably not more than 20%, particularly preferably not more than 10% and especially preferably 0% (inactivation of the serB gene) of the activity of the wild-type strain set at 100%. The SerB activity still measurable in the modified microorganism strain and based on the activity in the Wt train as a percentage is referred to as residual activity.

A microorganism strain having suppressed SerB activity is characterized by one or more of the following genetic modifications:
Deletion of the chromosomal gene encoding the enzyme SerB.
Introduction of a mutation into the chromosomal gene encoding the enzyme SerB in order to reduce the activity of the endogenous gene.
Substitution of the chromosomal gene encoding the enzyme SerB with a gene which is mutated in order to reduce the activity of the endogenous gene.
Introduction of a mutation into a regulatory region for the gene encoding the enzyme SerB in order to reduce endogenous enzyme activity.
Introduction of an antisense oligonucleotide complementary to the transcript of the gene encoding the enzyme SerB in order to inhibit the translation of the mRNA.

As described in the prior art, L-3-phosphoserine phosphatase enzyme activity (SerB activity) can be determined by enzymatic release of phosphate from L-3-phosphoserine, the released phosphate being quantitatively determined photometrically at 340 nm as a molybdate complex. The serB activity determined for the WT strain using this enzyme assay is set at 100% activity and the residual activity for a microorganism strain having suppressed SerB activity is measured under identical conditions.

Particularly preferably, the process for producing L-cysteic acid according to the invention is characterized in that the OPS used in the reaction is produced using a microorganism strain in which the chromosomal gene encoding the enzyme SerB has been deleted. This is also called a knockout of the serB gene. Preferably, the microorganism strain having suppressed SerB activity is in this case characterized in that the chromosomal nucleotide sequence of the serB gene comprising the complete serB cds encoding the enzyme SerB and flanking sequences up to 1000 nt 5′ upstream of the serB cds, comprising the sequence of the serB promoter, and 1000 nt 3′ downstream of the serB cds, comprising the sequence of the serB terminator, has been deleted. A particularly preferred deletion is the one described in example 1, the deletion of the serB cds encoding the enzyme SerB.

The microorganism strain having suppressed SerB activity is preferably selected from the families Corynebacteriaceae and Enterobacteriaceae, particularly preferably selected from the genera Corynebacterium, Pantoea and Escherichia and especially preferably selected from the species Pantoea ananatis and Escherichia coli. Very preferably, the microorganism strain having suppressed SerB activity for producing OPS for the reaction according to the invention is the strain E. coli K12 W3110.

A microorganism strain having suppressed SerB activity is preferably characterized by serine auxotrophy, i.e., the strain is not able itself to form the amino acid L-serine for growth. Auxotrophy can be overcome by addition of serine or glycine to the culture medium (growth medium), in each case either as pure substance or as a constituent of a complex media component such as yeast extract, peptone or tryptone, and as a mixture of pure substance and complex media component. Preference is given to a mixture of pure substance, selected from glycine and L-serine, and complex media component, including particular preference for a mixture of glycine and complex media component. It is especially preferred to add glycine to the growth medium.

The content of glycine as pure substance in the growth medium is preferably 0.1 g/L to 10 g/L, particularly preferably 0.2 g/L to 5 g/L and especially preferably 0.3 g/L to 2 g/L.

According to the prior art, the serB gene or part of the gene can be isolated and foreign DNA can be cloned into the serB gene, thereby interrupting the protein-defining open reading frame of the serB gene. A DNA construct suitable for the targeted inactivation of the serB gene can thus consist of a 5′ section of DNA which is homologous to the genomic serB gene, followed by a gene segment comprising the foreign DNA, followed by a 3′ section of DNA which is again homologous to the genomic serB gene.

The possible region in the serB gene for homologous recombination can thus comprise not just the region encoding O-phospho-L-serine phosphatase. The possible region can also comprise DNA sequences flanking the serB gene, namely in the 5′ region before the start of the coding region (gene transcription promoter) and in the 3′ region after the end of the coding region (gene transcription terminator), the modification of which by homologous recombination can lead to inactivation of the serB gene just like the modification of the coding region.

The foreign DNA is preferably a selection-marker expression cassette. It consists of a gene transcription promoter functionally linked to the actual selection marker gene, optionally followed by a gene transcription terminator. In this case, the selection marker also contains 5′ and 3′ flanking homologous sequences of the serB gene. Preferably, the selection marker contains 5′ and 3′ flanking homologous sequences of the serB gene that each have a length of at least 30 nucleotides, particularly preferably at least 50 nucleotides.

The DNA construct for inactivating the serB gene can thus, starting from the 5′ end, consist of a sequence homologous to the serB gene, followed by the expression cassette of the selection marker, for example selected from the class of the antibiotic resistance genes, and followed by a further sequence homologous to the serB gene. In a preferred embodiment, the DNA construct for inactivating the serB gene consists, starting from the 5′ end, of a sequence homologous to the serB gene of at least 30 nucleotides in length, especially preferably at least 50 nucleotides in length, followed by the expression cassette of the selection marker, selected from the class of the antibiotic resistance genes, and followed by a further sequence homologous to the serB gene of at least 30 nucleotides in length, especially preferably at least 50 nucleotides in length.

The selection marker genes are generally genes, the gene product of which enables the parent strain to grow under selective conditions under which the original parent strain cannot grow.

Preferred selection marker genes are selected from the group of the antibiotic resistance genes such as, for example, the ampicillin resistance gene, the tetracycline resistance gene, the kanamycin resistance gene, the chloramphenicol resistance gene or else the neomycin resistance gene. Other preferred selection marker genes allow parent strains having a metabolic defect (e.g., amino acid auxotrophies) to grow under selective conditions as a result of correction of the metabolic defect by expression of said selection marker genes. Lastly, another possibility is selection marker genes, the gene product of which chemically alters an inherently toxic compound for the parent strain and thus inactivates said compound (e.g., the gene of the enzyme acetamidase, which splits the compound acetamide, toxic for many microorganisms, into the nontoxic products acetate and ammonia). The ampicillin resistance gene, the tetracycline resistance gene, the kanamycin resistance gene and the chloramphenicol resistance gene are particularly preferred among the selection marker genes. The tetracycline resistance gene and the kanamycin resistance gene are especially preferred.

There are also systems based on homologous recombination that, in addition to targeted gene inactivation, also provide the option of removing the selection marker from the genome, thereby making it possible to produce double and multiple mutants.

Such a system is, for example, so-called Lambda Red technology, commercially available as the “Quick and Easy E. coli Gene Deletion Kit”, based on Red®/ET® technology from Gene Bridges GmbH (see “Technical Protocol, Quick & Easy E. coli Gene Deletion Kit, by Red®/ET® Recombination, Cat. No. K006, Version 2.3, June 2012” and literature cited therein, for example Datsenko and Wanner, Proc. Natl. Acad. Sci. USA 97 (2000): 6640-6645).

Example 1 of the present invention describes an example for producing a microorganism strain having suppressed SerB activity by deletion of the serB gene.

A microorganism strain having suppressed SerB activity produced using Red®/ET® technology may be suitable for extracellular production of OPS, as described for example for the strain E. coli W3110-ΔserB in the 2nd example of the present invention. OPS can accumulate both intracellularly and extracellularly, the amount of intracellularly accumulated OPS depending on the growth conditions (Steinfeld et al., see above). The culture conditions chosen in example 2 of the present invention allow the extracellular accumulation of OPS. The content of extracellular OPS is preferably at least 1 g/L, particularly preferably at least 3 g/L and especially preferably at least 6 g/L.

An advantage of the described process for biotechnological production of OPS is that OPS present extracellularly in a culture broth, as obtained for example in a growth as carried out in the 2nd example, can be used directly in the process according to the invention for producing L-cysteic acid as a source of OPS after removal of the particulate biomass, for example by centrifugation or filtration, preferably without further workup, purification or isolation steps, examples including extraction, adsorption, ion-exchange chromatography, precipitation and crystallization. This procedure is particularly economical and avoids isolating OPS. Especially preferably, the process for producing L-cysteic acid is therefore characterized in that use is made of OPS which comes from the cell culture supernatant of the growth of a microorganism strain having suppressed activity of the O-phospho-L-serine phosphatase (SerB enzyme) belonging to enzyme class EC 3.1.3.3.

In a particularly preferred embodiment, the process for producing L-cysteic acid is characterized in that, in addition to the CS enzyme used in the reaction, the OPS used in the reaction is produced biotechnologically, especially preferably by fermentation.

Preferably, the CS enzyme is produced by culture of a microorganism strain from the Enterobacteriaceae family which expresses the CS enzyme heterologously.

Said microorganism strain is also referred to as CS enzyme production strain, consisting of the host strain and of a gene construct for expression of the CS gene. Preferably, the process is characterized in that the CS enzyme is produced by growth of a microorganism strain from the genus Escherichia, particularly preferably from the species Escherichia coli, and especially preferably of the microorganism strain E. coli K12 JM105, which expresses the CS enzyme heterologously and very preferably in enzymatically active form.

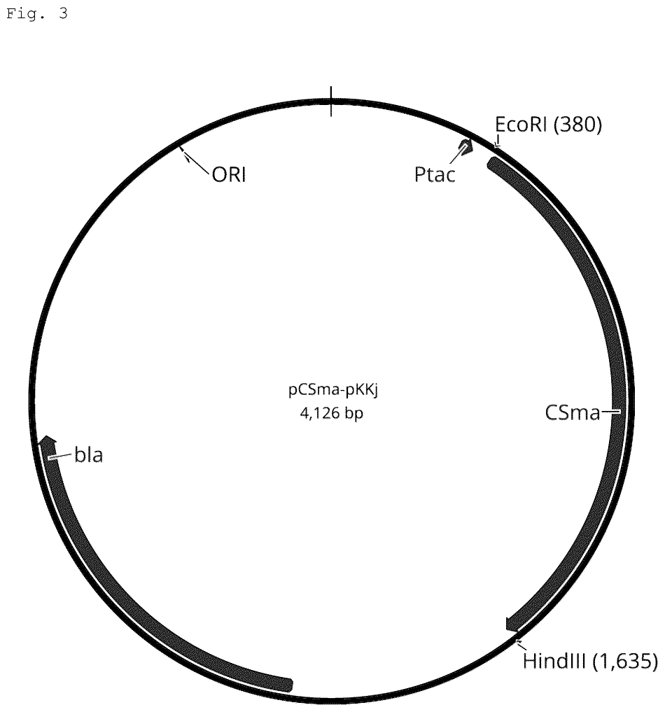

A preferred gene construct for expression of the CS gene is an expression vector in plasmid form, including particularly preferably the expression vector pCSma-pKKj disclosed in example 3 (FIG. 3).

Cysteate synthases are known as enzymes of coenzyme M biosynthesis by, for example, methanobacteria. Preferably, the process for producing L-cysteic acid is characterized in that the CS enzyme comes from Methanoscarcina acetivorans or the CS enzyme is a sequence homologous thereto, particular preference being given to the CS enzyme coming from Methanoscarcina acetivorans. Especially preferably, the coding DNA sequence is SEQ ID NO: 3, which encodes a protein having the amino acid sequence SEQ ID NO: 4, or a nucleotide sequence homologous thereto.

Homologous nucleotide sequences are to be understood to mean that the DNA sequences of these genes or sections of DNA are at least 80% identical, preferably at least 90% identical and particularly preferably at least 95% identical. Preferred homologous nucleotide sequences are the sequences of the genes from Methanocella paludicola (NCBI Gene ID: 8682885), Methanosarcina barkeri (NCBI Gene ID: 24822660), Methanoculleus marisnigri (NCBI Gene ID: 4845938) or nucleotide sequences homologous thereto.

The degree of DNA identity is determined by the “nucleotide blast” program, which can be found at http://blast.ncbi.nlm.nih.gov/ and is based on the blastn algorithm. The algorithm parameters used to align two or more nucleotide sequences were the default parameters. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1, −2; Gap Costs=Linear.

In a preferred embodiment, the process is characterized in that the CS enzyme has the amino acid sequence specified in SEQ ID NO: 4 or an amino acid sequence homologous thereto, wherein an amino acid sequence homologous to SEQ ID NO: 4 has a sequence identity of at least 50%, preferably at least 70% and especially preferably at least 80% in relation to SEQ ID NO: 4 and, at the same time, cysteate synthase enzyme activity. Cysteate synthase enzyme activity can be detected as defined above in a CS activity assay. Amino acid sequences of homologous CS enzymes can be found in the NCBI database (National Center for Biotechnology Information) using the search term “cysteate synthase” or using the subsidiary program “Protein BLAST” by input of the amino acid sequence SEQ ID NO: 4. Enzymes homologous to the CS enzyme from Methanoscarcina acetivorans are preferably selected from Methanocella paludicola (NCBI No.: WP_012900738.1), Methanolinea mesophila (NCBI No.: WP_245249687.1), Methanosarcina barkeri (NCBI No.: WP_011308449.1), Methanoculleus marisnigri (NCBI No.: WP_011842967.1).

A “Protein BLAST” search for genes for CS enzymes returns homologous protein sequences from a multitude of bacteria from the domain of the Archaebacteria, which also includes thermophilic organisms (growth at temperatures >50° C. up to 110° C.). The enzymes thereof usually likewise have activity optima >50° C. The invention also encompasses CS enzymes from the domain of the Archaebacteria having an activity optimum >50° C.

Protein sequences are compared using the “Protein BLAST” program at http://blast.ncbi.nlm.nih.gov/. This program uses the blastp algorithm. The algorithm parameters used to align two or more protein sequences were the default parameters. The default general parameters are: Max target sequences=100; Short queries=“Automatically adjust parameters for short input sequences”; Expect Threshold=10; Word size=3; Automatically adjust parameters for short input sequences=0. The default scoring parameters are: Matrix=BLOSUM62; Gap Costs=Existence: 11 Extension: 1; Compositional adjustments=Conditional compositional score template adjustment.

Preferably, the process is characterized in that the CS enzyme is not a fusion protein. The term “fusion protein” means that the DNA sequence encoding a protein or part of a protein is fused in frame in the laboratory with one or more DNA sequences encoding a further protein or part of a further protein and thus encodes a modified (extended) protein that does not occur in nature. The fused DNA sequences can be attached at the 5′ end, at the 3′ end, or at both the 5′ end and the 3′ end. It is also conceivable to insert the fused DNA sequence within the sequence encoding the protein (e.g., as a connection between two domains of a protein). The fusion protein is also referred to as a hybrid or hybrid enzyme. This means that the protein according to the invention having cysteate synthase enzyme activity is only encoded by the cds of the gene in question and the CS cds is not extended by sequences additionally added to the CS cds. A protein having a cds which has been fused with a nucleotide sequence which encodes a protein sequence which is cleaved off again during protein biosynthesis or during posttranslational modification, for example an export signal sequence mediating protein secretion, is not covered by the term “fusion protein”. The term “fusion protein” in the context of the invention always refers to the mature protein.

In the context of the invention, “the production of the CS enzyme by growth of a CS enzyme production strain” refers to the production of the CS enzyme as an enzymatically active protein without refolding and preferably without recourse to expression as a fusion protein. Particular preference is given to the production of the CS enzyme from M. acetivorans by growth of a CS enzyme production strain as an enzymatically active protein without refolding and without recourse to expression as a fusion protein, wherein the production strain is produced by using a host strain of the species Escherichia coli. Especially preferred is the production of the CS enzyme from M. acetivorans by growth of the production strain E. coli JM105×pCSma-pKKj described in example 3.

In the process according to the invention, the CS enzyme obtained by growth of the production strain can be used either as a culture broth without further workup or as a cell suspension after reisolation of the cells from the culture broth, for example by centrifugation or filtration. Furthermore, the CS enzyme can be used in the form of a cell homogenate after mechanical disruption of the cell suspension or in the form of chemically permeabilized cells (e.g., by chloroform) or else as a cell extract after removal of particulate constituents from the cell homogenate or else as an enzyme purified by chromatography, for example.

Preference is given to the use of the CS enzyme as a culture broth, especially preferably fermenter broth, without further workup, as a cell suspension after reisolation of the cells from the culture broth or else as a cell homogenate after mechanical disruption of the cell suspension or in the form of chemically permeabilized cells (e.g., by chloroform).

Particular preference is given to the use of the CS enzyme as a cell suspension after reisolation of the cells from the culture broth or as a cell homogenate, especially preferably as a cell homogenate.

In a preferred embodiment, growth of a CS enzyme production strain is followed by producing CS enzyme as a cell homogenate and using said homogenate directly in the biotransformation process according to the invention as CS enzyme. One possible preferred embodiment is disclosed in example 3 of the present invention.

In principle, all conceivable salts of sulfurous acid are suitable for the reaction in the process for producing L-cysteic acid, including the known salts Na₂SO₃, K₂SO₃, (NH₄)₂SO₃, NaHSO₃ (or its anhydride Na₂S₂O₅) or KHSO₃. It is also conceivable to use gaseous sulfur dioxide, the anhydride of sulfurous acid, which can be introduced into the reaction batch, where it is hydrated to sulfurous acid H₂SO₃ and, depending on the pH, is in an equilibrium with the deprotonated forms HSO₃⁻ and SO₃²⁻.

It is preferred to use Na₂SO₃, K₂SO₃, (NH₄)₂SO₃, NaHSO₃ (or its anhydride Na₂S₂O₅), KHSO₃, particularly preferred to use Na₂SO₃, NaHSO₃ (or its anhydride Na₂S₂O₅) and (NH₄)₂SO₃ and especially preferred to use Na₂SO₃ and NaHSO₃ (or its anhydride Na₂S₂O₅). In a very preferred embodiment, the salt of sulfurous acid used in the process for producing L-cysteic acid is Na₂SO₃ or NaHSO₃ (or its anhydride Na₂S₂O₅).

According to equation (1), the reaction of OPS to form L-cysteic acid releases stoichiometric amounts of phosphoric acid, which may lead to a decrease in pH in the batch as the reaction proceeds. Since an excessively low pH affects the activity of the CS enzyme, it is necessary to prevent an excessively large drop in pH. This can be done passively by a suitable highly concentrated buffer in the batch or can be achieved actively by a measurement and control unit.

Preference is given to active pH control (as described in example 6 for example) by a measurement and control unit which, in the event of deviation of the pH from the target value, restores the desired pH by metered addition of an alkaline solution or acid (so-called pH-stat method).

The reaction temperature is chosen between 5° C. and 80° C. Preference is given to a reaction temperature between 10° C. and 60° C., particular preference to between 15° C. and 50° C., and especial preference to between 20° C. and 40° C.

The reaction can be carried out at a pH between 4.0 and 9.0, preferably at a pH between 5.0 and 8.5, particularly preferably at a pH between 5.5 and 8.0 and especially preferably at a pH between 6.0 and 7.5.

The solvent used for the process for producing L-cysteic acid is preferably water.

The process according to the invention for producing L-cysteic acid can be carried out in discontinuous operation or continuous operation. In discontinuous operation (batch operation), all reactants are added to the batch in the course of the reaction and the batch is worked up after the reaction has ended. In continuous operation, the CS enzyme is charged as a stationary phase, for example immobilized in a membrane reactor or on a support, and the substrate OPS and a salt of sulfurous acid are metered in as a mobile phase. The contact time of the mobile phase with the stationary phase is set such that the substrate OPS can completely react with the salt of sulfurous acid to form the product L-cysteic acid. Preference is given to discontinuous (batch) operation.

The concentration of the salt of sulfurous acid in the batch is preferably chosen such that it is at least in equimolar concentration to OPS, preferably at least in 1.5-fold molar excess, particularly preferably in at least 2-fold molar excess and especially preferably in at least 5-fold molar excess to OPS.

The OPS concentration in the batch is preferably at least 80 mg/L, particularly preferably at least 1 g/L and especially preferably at least 5 g/L.

Preferably, the process for producing L-cysteic acid is characterized in that the molar yield of L-cysteic acid based on the molar amount of OPS used is at least 60%, particularly preferably at least 80% and especially preferably at least 90%.

The process according to the invention is suitable for use on an industrial scale. Preference is given to a batch volume >10 ml, particular preference to >1 L and especial preference to >100 L.

Contrary to the prior art, it has been found that, surprisingly, CS enzyme heterologously produced recombinantly in a CS enzyme production strain is enzymatically active in a cell homogenate without refolding (renaturation) and preferably without recourse to a fusion partner and is suitable in a previously unknown biotransformation for efficient production of L-cysteic acid. For this purpose, OPS produced by cultivation, for example by fermentation, and from the cell culture supernatant of the growth of an OPS production strain having preferably suppressed activity of the SerB enzyme or commercially available OPS can be reacted with a salt of sulfurous acid according to equation (1).

Preference is furthermore given to a process for producing taurine, characterized in that the L-cysteic acid produced according to the invention is decarboxylated. L-Cysteic acid is decarboxylated to taurine according to equation (2):

L-Cysteic acid->Taurine+CO₂ (2)

Especially preferably, the process for producing taurine is characterized in that the L-cysteic acid produced in the process according to the invention is used further directly, i.e., without further workup, purification or isolation steps, for production of taurine, as disclosed for example in examples 8 and 9 of the present invention.

The decarboxylation of L-cysteic acid to taurine can be effected chemically or under enzymatic catalysis in a biotransformation. Thermal decarboxylation at high temperatures under metal catalysis, which is not considered sustainable, is known, but it has the disadvantage of being energy-intensive and producing a high proportion of by-products. Preferably, the decarboxylation reaction is a biotransformation with enzymatic decarboxylation of L-cysteic acid to taurine.

To produce taurine by enzymatic decarboxylation, the L-cysteic acid produced in the process according to the invention can be used directly in the form of the reaction batch without further workup steps. However, it is also possible to remove particulate biomass from the reaction batch, for example by centrifugation, prior to the enzymatic decarboxylation or to isolate L-cysteic acid from the reaction batch beforehand.

Preference is given to the direct use of the L-cysteic acid-containing reaction batch or to the use of the L-cysteic acid-containing reaction batch after removal of particulate biomass.

Particular preference is given to the direct use of the L-cysteic acid-containing reaction batch without further workup steps, as disclosed for example in example 8 for production of taurine.

Suitable for enzymatically catalyzed decarboxylation of L-cysteic acid to taurine according to equation (2) are enzymes from the class of L-cysteine sulfinic acid decarboxylases (CSAD, EC 4.1.1.29), aspartate 1-decarboxylases (EC 4.1.1.11) or glutamate decarboxylases (EC 4.1.1.15).

Preferably, the process for producing taurine is characterized in that the decarboxylation is effected by means of a cysteine sulfinic acid decarboxylase (CSAD enzyme) belonging to enzyme class EC 4.1.1.29. CSAD enzymes are known to decarboxylate L-cysteine sulfinic acid to hypotaurine according to equation (3).

L-Cysteine sulfinic acid->Hypotaurine+CO₂ (3)

To varying degrees, these enzymes are also capable of decarboxylating L-cysteic acid as substrate to taurine. As shown, inter alia, in examples 8 and 9, the CSADcc enzyme from Cyprinus carpio (carp), for example, is suitable for decarboxylating L-cysteic acid to taurine according to equation (2).

CSAD enzymes are mainly found in Metazoa (multicellular animals), including in mammals, for example human (Homo sapiens), cattle (Bos taurus), rat (Rattus norvegicus), mouse (Mus musculus), but also in fishes, for example carp (Cyprinus carpio). Enzymes having CSAD activity can also be found in single-cell organisms, such as in algae, for example from the genus Synechococcus, and also in bacteria or fungi.

Preference is given to CSAD enzymes from mammals, selected from human (Homo sapiens), cattle (Bos taurus), rat (Rattus norvegicus) or mouse (Mus musculus), and from fishes, for example from carp (Cyprinus carpio).

Particular preference is given to CSAD enzymes from human (Homo sapiens), rat (Rattus norvegicus) or carp (Cyprinus carpio).

Especially preferably, the CSAD enzyme comes from carp (Cyprinus carpio) and is referred to as CSADcc. The DNA sequence forming the basis of the CSADcc amino acid sequence is accessible in the NCBI database under the GenBank sequence ID: AB220585.1 (cds: nt 82-1584). Preferably derived from the corresponding amino acid sequence is a CSADcc cds DNA sequence codon-optimized for expression in a specifically chosen microorganism (such as E. coli) (e.g., specified in SEQ ID NO: 5, nt 31-1530, for E. coli, encoding a protein having the amino acid sequence SEQ ID NO: 6). Publicly available software programs are available for codon optimization, such as the Eurofins Genomics GENEius software used in example 7.

Preferably, the process for producing taurine is characterized in that the amino acid sequence of the CSAD enzyme is SEQ ID NO. 6.

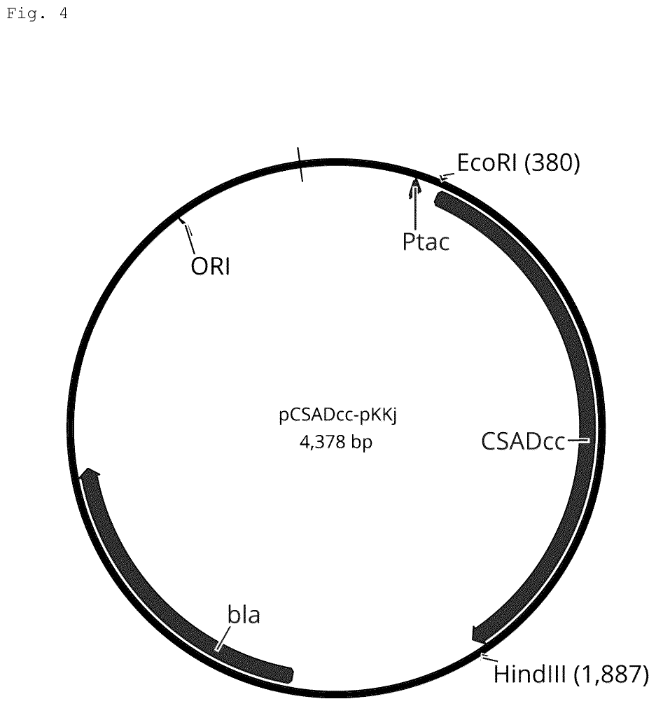

Preferably, the CSAD enzyme, particularly preferably CSADcc, is produced recombinantly by a microorganism production strain. The production of the CSAD enzyme by recombinant production in an E. coli production strain is disclosed in example 7, for example. The CSAD cds is cloned into an expression vector, for example the vector pKKj (see example 3), in a known manner and a gene construct, for example pCSADcc-pKKj (FIG. 4), is produced. A production strain is produced by transforming the gene construct containing the CSAD cds, for example pCSADcc-pKKj, into a microorganism host strain, for example the strain E. coli JM105, in a likewise known manner and using the resultant production strain, for example E. coli JM105×pCSADcc-pKKj, for production of the CSAD enzyme in a likewise known manner. The CSAD enzyme can be produced on a shake flask scale for laboratory purposes (as described for example in example 7), or by fermentation in a known manner.

CSAD enzymes contain pyridoxal phosphate (PLP, CAS No. 54-47-7) as a cofactor. Supplementing the growth medium or biotransformation batches for conversion of L-cysteic acid to taurine with PLP therefore offers one way of achieving process improvement. Since PLP belongs to the vitamin B6 family, supplementation with other members of the vitamin B6 family, such as pyridoxine (CAS No. 65-23-6), pyridoxal (CAS No. 66-72-8) or pyridoxamine (CAS No. 85-87-0), is a suitable alternative for process improvement.

The CSAD enzyme, preferably CSADcc, obtained by growth in a shake flask or by fermentation can be used either as a culture broth without further workup or as a cell suspension after reisolation of the cells by, for example, centrifugation. Furthermore, the CSAD enzyme, preferably CSADcc, can be used in the form of a cell homogenate after mechanical disruption of the cell suspension or in the form of chemically permeabilized cells (e.g., by chloroform) or else as a cell extract after removal of particulate constituents from the cell homogenate or else as an enzyme purified by chromatography, for example. Preference is given to the use of the CSAD enzyme as a cell suspension after reisolation of the cells from the culture broth, as described for example in examples 7 and 8.

The biotransformation of L-cysteic acid to taurine by the CSAD enzyme is carried out under pH and temperature conditions that allow efficient decarboxylation of L-cysteic acid to taurine. Preference is given to a pH range between pH 5.0 and 9.0 and a temperature range between 20° C. and 70° C. at which the biotransformation is carried out.

The biotransformation for production of taurine from L-cysteic acid can be carried out in discontinuous or continuous operation. In discontinuous operation (batch operation), all reactants are added to the batch in the course of the reaction and the batch is worked up after the reaction has ended. In continuous operation, the CSAD enzyme is charged as a stationary phase, for example immobilized in a membrane reactor or on a support, and the substrate L-cysteic acid is metered in as a mobile phase. The contact time of the mobile phase with the stationary phase is set such that the substrate L-cysteic acid can completely react to form the product taurine. Preference is given to discontinuous (batch) operation.

The concentration of L-cysteic acid in the biotransformation for production of taurine is preferably at least 80 mg/L, particularly preferably at least 1 g/L and especially preferably at least 5 g/L.

Preferably, the process for producing taurine is characterized in that the molar yield of taurine based on the molar amount of L-cysteic acid used is at least 60%, preferably at least 80%, particularly preferably at least 90% and especially preferably at least 95%.

Preferably, the process steps for production of L-cysteic acid (biotransformation 1) and for production of taurine proceed sequentially, i.e., one after the other. In an alternatively preferred embodiment, the process for producing taurine is characterized in that all the process steps take place in one reaction batch.

If all the process steps take place in one reaction batch, the process is also called a one-pot process or one-pot reaction.

Example 9 of the present invention discloses a way of carrying out such a one-pot reaction, in which the inventive biotransformation of OPS to L-cysteic acid according to equation (1) and the biotransformation of L-cysteic acid to taurine according to equation (2) take place simultaneously, i.e., in one reaction batch, with OPS being reacted with a sulfite (salt of sulfurous acid) in the presence of the CS enzyme and CSAD enzyme. L-cysteic acid is formed in the first reaction and it is decarboxylated “in situ” to taurine by CSAD enzyme. The product distribution of L-cysteic acid and taurine is determined by the activity of the CS enzyme in relation to that of the CSAD enzyme. With sufficient metered addition of the CSAD enzyme, the L-cysteic acid can be converted quantitatively to taurine. Preference is given to a process in which the OPS used is converted to L-cysteic acid and taurine, and the total molar yield of L-cysteic acid and taurine is more than 60%, particularly preferably more than 70% and especially preferably more than 80%.

Carrying out the process for producing L-cysteic acid and the decarboxylation to taurine in one reaction batch is of particular interest when considering economic viability.

It is also conceivable that, in the context of a metabolic engineering approach, the genes for the cysteate synthase, preferably CSma, and the L-cysteine sulfinic acid decarboxylase, preferably CSADcc, are expressed in the OPS production strain and taurine is produced by growth of such a production strain in the presence of a sulfur source, preferably a sulfite (salt of sulfurous acid). Likewise, it is conceivable that the genes for the cysteate synthase, preferably CSma, and the L-cysteine sulfinic acid decarboxylase, preferably CSADcc, are jointly expressed in one strain and the cells from the growth of said strain are reacted with OPS in the presence of a sulfite to produce taurine as the end product.

Preference is given to a biotransformation process for producing taurine, in which the process components OPS, CS enzyme and CSAD enzyme are produced separately.

Taurine can be either used further directly without further workup steps or else enriched or purified by means of known methods. Such methods are known to a person skilled in the art, for example from methods for isolating amino acids. Examples include filtration, centrifugation, extraction, adsorption, ion-exchange chromatography, precipitation, crystallization.

The invention further provides for the use of the L-cysteic acid formed in a process for producing taurine.

Compared to the known chemical processes for producing taurine from fossil raw materials, the use of the L-cysteic acid produced in the process according to the invention to produce taurine allows a biotechnological process starting from plant raw materials. A biotechnological process for producing taurine is of particular interest for applications in the food sector, feed sector or cosmetics sector because of the sustainable production process.

The invention will be further illustrated by the following examples without being restricted by them:

### Example 1: Production of a serB Deletion Mutant in Escherichia coli

The strain used was Escherichia coli K12 W3110 (commercially available under the strain number DSM 5911 from the DSMZ: Deutsche Sammlung von Mikroorganismen und Zellkulturen GmbH [German Collection of Microorganisms and Cell Cultures]). The target of gene inactivation was the coding sequence of the serB gene from E. coli. The DNA sequence of the cds of the serB gene from E. coli K12 (SEQ ID NO: 1, nt 67 to nt 1032), encoding a protein having the amino acid sequence SEQ ID NO: 2, is accessible in the NCBI (National Center for Biotechnology Information) gene database with the gene ID 948913.

The E. coli serB gene was inactivated using Red®/ET® technology from Gene Bridges GmbH as detailed below (described in the user manual of the “Quick and Easy E. coli Gene Deletion Kit”, see “Technical Protocol, Quick & Easy E. coli Gene Deletion Kit, by Red®/ET® Recombination, Cat. No. K006, Version 2.3, June 2012 and literature cited therein, for example Datsenko and Wanner, Proc. Natl. Acad. Sci. USA 97 (2000): 6640-6645). To this end, use was made of the plasmids pKD13, pKD46 and pCP20:
The 3.4 kb plasmid pKD13 (FIG. 1) is disclosed in the “GenBank” gene database under the accession number AY048744.1.
The 6.3 kb plasmid pKD46 (FIG. 2) is disclosed in the “GenBank” gene database under the accession number AY048746.1.
The 9.4 kb plasmid pCP20 is disclosed in Cherepanov and Wackernagel, Gene 158 (1995): 9-14.

To inactivate the serB gene in E. coli W3110 by homologous recombination using the Lambda Red system, the following steps were carried out:
1. E. coli W3110 was transformed with the plasmid pKD46 (so-called “Red Recombinase” plasmid, FIG. 2) and an ampicillin-resistant clone was isolated, referred to as W3110×pKD46.
2. A serB-specific DNA fragment suitable for inactivation thereof was produced in a PCR reaction (“Phusion™ High-Fidelity” DNA polymerase, Thermo Scientific™) with DNA of the plasmid pKD13 (FIG. 1) and the primers serb-1f (SEQ ID NO: 7) and serb-2r (SEQ ID NO: 8). Formed in the PCR reaction was a 1.4 kb PCR product which contained at both the 5′ end and the 3′ end a 30 nt section of DNA that was specific for the serB gene from E. coli W3110. Furthermore, the PCR product contained the expression cassette of the kanamycin resistance gene contained in pKD13 and, flanking the 5′ and 3′ ends of the kanamycin expression cassette, so-called “FRT direct repeats” (referred to as “FRT1” and “FRT2” in FIG. 1), short sections of DNA that were used as a recognition sequence for “FLP recombinase” (contained on the plasmid pCP20) in a later working step for removal of the antibiotic marker kanamycin.
   Primer serb-1f contained 30 nucleotides (nt) from the 5′ region of the serB gene (nt 67-96 in SEQ ID NO: 1) followed by 20 nt specific for the plasmid pKD13 (referred to as “pr-1” in FIG. 1).
   Primer serb-2r contained 30 nt from the 3′ region of the serB gene (nt 1006-1035 in SEQ ID NO: 1, in reverse complementary form) followed by 20 nt specific for the plasmid pKD13 (referred to as “pr-2” in FIG. 1).
3. The 1.4 kb PCR product was isolated and treated with the restriction endonuclease Dpn I, which is familiar to a person skilled in the art and which only cuts methylated DNA, in order to remove residual pKD13 plasmid DNA. Nonmethylated DNA from the PCR reaction is not degraded.
4. The 1.4 kb PCR product, which is specific for the serB gene and contains an expression cassette for the kanamycin resistance gene, was transformed into E. coli W3110×pKD46 and kanamycin-resistant clones were isolated on LBkan-glycine plates at 30° C. LBkan-glycine plates contained LB medium (10 g/L tryptone from GIBCO™, 5 g/L yeast extract from BD Biosciences, 5 g/L NaCl), 1.5% agar, 15 mg/L kanamycin (Sigma-Aldrich) and 1 g/L glycine (Sigma-Aldrich).
5. Four of the kanamycin-resistant clones obtained were purified on LBkan-glycine plates (i.e., isolation of a clone by singularization) and checked in a PCR reaction to determine whether the kanamycin-resistance cassette had been correctly integrated in the serB gene.
   The genomic DNA used for the PCR reaction (“Phusion™ High-Fidelity” DNA Polymerase, Thermo Scientific™) was isolated using a DNA isolation kit (Qiagen) from cells from the growth of kanamycin-resistant clones of E. coli W3110×pKD46 in LBkan-glycine medium (10 g/L tryptone, 5 g/L yeast extract, 5 g/L NaCl, 15 mg/L kanamycin, 1 g/L glycine). Genomic DNA of the E. coli W3110 wild-type strain was used as control. The primers used for the PCR reaction were serb-3f (SEQ ID NO: 9, 5′ flanking the serB gene, nt 1-22 in SEQ ID NO: 1) and serb-4r (SEQ ID NO: 10, 3′ flanking the serB gene, nt 1066-1085 in SEQ ID NO 1, in reverse complementary form).
   E. coli W3110 wild-type DNA yielded a DNA fragment of 1.1 kb in the PCR reaction, as expected for the intact gene. By contrast, the four kanamycin-resistant clones under study yielded a DNA fragment of approx. 1.6 kb in the PCR reaction, as expected if the 1.4 kb PCR product had been integrated in the serB gene at the sites defined by the primers serb-1f and serb-2r. This result showed that the kanamycin resistance gene had been successfully integrated at the locus of the serB gene and that the serB gene had thus been inactivated. A clone having an inactivated serB gene was selected and treated at 42° C. to remove the temperature-sensitive plasmid pKD46, thereby making the strain ampicillin-sensitive again. The strain was designated W3110-ΔserB::kan.
6. To eliminate the kanamycin selection marker, W3110-ΔserB::kan was transformed with the plasmid pCP20 and transformants were selected at 30° C. The 9.4 kb vector pCP20 is disclosed in Cherepanov and Wackernagel (1995), Gene 158: 9-14. Present on the vector pCP20 is the gene of FLP recombinase. FLP recombinase recognizes the FRT sequences flanking the expression cassette of the kanamycin resistance gene and brings about the removal of the kanamycin expression cassette. To this end, the clones obtained at 30° C. were incubated at 37° C. Under these conditions, the expression of FLP recombinase was induced and the replication of the pCP20 vector was suppressed.
   The result of this step were clones in which the serB gene had been inactivated and which had regained sensitivity to kanamycin (so-called “curing” of the antibiotic selection marker). The removal of the kanamycin cassette from the genome of the ΔserbB mutants allows the introduction of further mutations in order to produce double or multiple mutants.
   W3110-ΔserB::kan regained kanamycin sensitivity after the treatment with the pCP20 plasmid, which was checked as follows:
      by plating on LB-glycine und LBkan-glycine plates:
      Growth on LB-glycine plates was positive, whereas growth was no longer observed on LBkan-glycine plates, which indicated the successful removal of the kanamycin cassette from the genome.
      by PCR reaction:
      To this end, genomic DNA was isolated from the kanamycin-sensitive clones (Qiagen DNA isolation kit) and used in a PCR reaction (“Phusion™ High-Fidelity” DNA Polymerase, Thermo Scientific™) using the primers serb-3f (SEQ ID NO: 9) and serb-4r (SEQ ID NO: 10). E. coli W3110 wild-type DNA yielded a DNA fragment of 1.1 kb in the PCR reaction, as expected for the intact serB gene. By contrast, the kanamycin-sensitive clone yielded a DNA fragment of approx. 250 nt in the PCR reaction, which corresponded to the expected size of the 5′ and 3′ fragments of the inactivated serB gene remaining after homologous recombination.
   The strain isolated from this step was designated E. coli W3110-ΔserB. This strain is distinguished by the fact that it contained an inactivated serB gene and that said strain regained sensitivity to the antibiotic kanamycin.

### Example 2: Production of OPS

### Production in a Shake Flask:

OPS was produced by growth of the strain E. coli W3110-ΔserB in a shake flask. For comparison, OPS production in the WT strain E. coli W3110 was analyzed. As a preculture for cultivation in a shake flask, the strains E. coli W3110 and E. coli W3110-ΔserB were each inoculated in 3 ml of LB-glycine medium (10 g/L tryptone, 5 g/L yeast extract, 10 g/L NaCl, 0.1 g/L glycine) and incubated in a shaker at 30° C. and 135 rpm for 16 h.

Main culture: Thereafter, a portion of the respective preculture was transferred to a 300 ml Erlenmeyer flask (baffled) containing 30 ml of SM1 medium containing 15 g/L glucose, 5 mg/L vitamin B1 (Sigma-Aldrich), 0.1 g/L each of the amino acids L-isoleucine, D,L-methionine and L-threonine, and 0.5 g/L glycine (all Sigma-Aldrich).

Composition of the SM1 medium: 12 g/L K₂HPO₄, 3 g/L KH₂PO₄, 5 g/L (NH₄)₂SO₄, 0.3 g/L MgSO₄×7 H₂O, 0.015 g/L CaCl₂)×2 H₂O, 0.002 g/L FeSO₄×7 H₂O, 1 g/L Na₃ citrate×2 H₂O, 0.1 g/L NaCl; 1 ml/L trace element solution.

Composition of the trace element solution: 0.15 g/L of Na₂MoO₄·2H₂O, 2.5 g/L of H₃BO₃, 0.7 g/L of CoCl₂·6H₂O, 0.25 g/L of CuSO₄·5H₂O, 1.6 g/L of MnC1₂·4H₂O, 0.3 g/L of ZnSO₄·7H₂O.

The main cultures were inoculated with enough preculture to establish an initial cell density OD₆₀₀/ml (optical density of the main culture, measured at 600 nm) of 0.3/ml in each case. Starting from this, the 30 ml batches were incubated at 30° C. and 135 rpm for 24 h.

After 24 h, samples were taken and the cell density OD₆₀₀/ml and the content of OPS were determined both in the culture supernatant and in the cell pellet. To this end, 2 ml of cell culture were centrifuged at 13 000 rpm for 5 min in each case (Heraeus™ Fresco™ 21 centrifuge). The cell culture supernatants were directly analyzed by HPLC for the content of OPS. The cell pellets were resuspended in 2 ml of H₂O in each case and cell extracts were prepared. This was accomplished by using the FastPrep-24™ 5G cell homogenizer from MP Biomedicals. In each case, 2×1 ml of cell pellets suspended in H₂O were disrupted in manufacturer-assembled 1.5 ml tubes containing glass beads (“Lysing Matrix B”) (3×20 sec at a shaking frequency of 6000 rpm with a pause between the intervals of 30 sec in each case). In each case, the cell homogenates obtained were combined and centrifuged at 13 000 rpm for 5 min in order to prepare a cell extract. The cell extract were analyzed by HPLC for the content of OPS. The result is summarized in Table 1.

TABLE 1
Growth of the strains E. coli W3110 and E. coli
W3110-ΔserB and HPLC analysis of the OPS content
of cell culture supernatant and cell extract

| | | OPS concentration | OPS concentration |
|---|---|---|---|
| | Cell density | in supernatant | in cell extract |
| Strain | OD₆₀₀/ml | mg/L | mg/L |
| W3110 | 5.1 | 0.0 | 0.0 |
| W3110-ΔserB | 6.6 | 172.2 | 0.0 |

HPLC analysis of OPS, L-cysteic acid and taurine: For quantitative determination of the compounds analyzed in the examples, an HPLC method calibrated respectively for OPS, L-cysteic acid and taurine was employed; all reference substances used for calibration were commercially available (Sigma-Aldrich). An Agilent 1260 Infinity II HPLC system was used, which was equipped with a unit from the same manufacturer for pre-column derivatization with o-phthaldialdehyde (OPA derivatization) as is known from the analysis of amino acids. For detection of the OPA-derivatized products of OPS, L-cysteic acid and taurine, the HPLC system was equipped with a fluorescence detector. The detector was set to an excitation wavelength of 330 nm and an emission wavelength of 450 nm. Also used was a Luna® C18(2) column from Phenomenex, length 250 mM, internal diameter 4.6 mm, particle size 5 μm, thermally equilibrated at 40° C. in a column oven.

Eluent A: 25 mM Na phosphate, pH 6.0. Eluent B: methanol. The separation was carried out in gradient mode: 1%-15% eluent B over 0-10 min, followed by 15% eluent B over 15 min, at a flow rate of 1.0 ml/min. Retention time of L-cysteic acid: 6.95 min. Retention time of OPS: 7.65 min. Retention time of taurine: 21.9 min.

### Production of OPS by Fermentation:

OPS was produced by fermentation of the strain E. coli W3110-ΔserB.

### Preculture 1:

20 ml of LB-glycine medium were inoculated with the strain E. coli W3110-ΔserB in a 100 ml Erlenmeyer flask and incubated on a shaker (150 rpm, 32° C.) for 7 h.

### Preculture 2:

Thereafter, the entire preculture 1 was transferred to 100 ml of SM1 medium supplemented with 10 g/L glucose, 10 g/L yeast extract, 0.3 g/L D,L-methionine, 1 g/L glycine and 5 mg/L vitamin B1. The culture was shaken in an Erlenmeyer flask (1 L volume) at 32° C. for 17 h at 150 rpm (Infors incubator shaker). Following this incubation, the cell density OD₆₀₀/ml was 5.7/ml.

Main culture:

Fermentation was carried out in a “DASGIP© Parallel Bioreactor System for Microbiology” fermenter from Eppendorf. Culture vessels with a total volume of 1.8 L were used. The fermentation medium (600 ml) contained 10 g/L glucose, 5 g/L yeast extract, 5 g/L (NH₄)₂SO₄, 5 g/L KH₂PO₄, 0.5 g/L NaCl, 0.225 g/L CaCl₂×2 H₂O, 1.2 g/L MgSO₄×7 H₂O, 0.075 g/L FeSO₄×7 H₂O, 1 g/L Na₃ citrate×2 H₂O, 1 g/L glycine, 1 g/L L-threonine, 0.018 g/L vitamin BI, 0.09 g/L vitamin B6 and 10 ml of trace element solution (see the section on shake flask growth).

The pH in the fermenter was initially adjusted to 7.0 by pumping in a 25% NH₄OH solution. During the fermentation, the pH was maintained at a value of 7.0 by automatic correction with 25% NH₄OH or 4 M H₃PO₄. Foam control was achieved by automatic metered addition of 4% v/v Struktol J673 in H₂O (Schill & Seilacher). For inoculation, 60 ml of preculture 2 were pumped into the fermenter vessel. The initial volume was therefore about 660 ml. The cultures were initially stirred at 400 rpm and aerated with compressed air sterilized via a sterile filter at an aeration rate of 2 vvm (volume of air per volume of culture medium per minute). Under these starting conditions, the oxygen probe was calibrated to 100% saturation prior to inoculation.

The target value for the O₂ saturation during the fermentation was set to 30%. After the O₂ saturation had fallen below the target value, a regulation cascade was started in order to bring the O₂ saturation back up to the target value. This involved first increasing the gas supply continuously (to a maximum of 5 vvm) and then increasing the stirring speed continuously (to a maximum of 1600 rpm). The fermentation was carried out at a temperature of 32° C.

Once the glucose content in the fermenter had fallen from an initial 10 g/L to approx. 2 g/L, a 56% (w/w) glucose solution was continuously metered in. The feeding rate was adjusted such that the glucose concentration in the fermenter no longer exceeded 2 g/L from then on. Glucose was determined using a glucose analyzer from YSI (Yellow Springs, Ohio, USA). 23 h after the start of fermentation, the fermentation batch was augmented by 3.5 ml of a 200 g/L glycine solution in H₂O.

The fermentation time was 53 h. 23 h, 30 h, 47 h and 53 h after the start of fermentation, samples were taken from the fermentation batch and cell density OD₆₀₀/ml was determined from one aliquot. In each case, a further aliquot was incubated at 80° C. for 5 min and centrifuged and, from the cell culture supernatant, the content of OPS was determined by HPLC. Cell density and OPS content are summarized in Table 2.

TABLE 2
Time course of cell density and OPS content in the
fermentation of the strain E. coli W3110-ΔserB

| | | OPS concentration in cell |
|---|---|---|
| Time | Cell density | culture supernatant |
| [h] | OD₆₀₀/ml | [g/L] |
| 23 | 58.0 | 2.7 |
| 30 | 64.0 | 3.5 |
| 47 | 80.0 | 6.5 |
| 53 | 110.0 | 6.9 |

### Example 3: Production of CSma Enzyme

Cysteate synthase from Methanosarcina acetivorans (CSma) was used. The amino acid sequence of the CSma enzyme is accessible in the NCBI database under the accession ID WP_048066469. The amino acid sequence was used to derive a DNA sequence codon-optimized for expression in E. coli (publicly available Eurofins Genomics GENEius software), which was synthetically produced (Eurofins Genomics). The synthetically produced DNA had the sequence disclosed in SEQ ID NO: 3 and contained the cds of the gene, referred to hereinafter as CSma cds (SEQ ID NO: 3), encoding a protein having the amino acid sequence disclosed in SEQ ID NO: 4 and referred to as CSma. For cloning purposes, the synthetically produced DNA contained at the 5′ end an EcoRI cleavage site and at the 3′ end a HindIII cleavage site.

The vector pCSma-pKKj suitable for recombinant expression of the CSma cds (FIG. 3) was produced by cutting the synthetically produced DNA using EcoRI and HindIII and cloning it in a known manner as an EcoRI/HindIII fragment into the vector pKKj cut using EcoRI and HindIII. The expression vector pKKj, disclosed in EP2670837A1 (Wacker application), is a derivative of the expression vector pKK223-3. The DNA sequence of pKK223-3 is disclosed in the GenBank gene database under the accession number M77749.1. From the 4.6 kb plasmid, approx. 1.7 kb (bp 262-1947 of the DNA sequence disclosed in M77749.1) were removed, thereby yielding the 2.9 kb expression vector pKKj.

The CSma cds was expressed in E. coli by transforming the vector pCSma-pKKj in a known manner into the strain E. coli K12 JM105. The strain E. coli K12 JM105 is commercially available under the strain number DSM 3949 from the DSMZ-German Collection of Microorganisms and Cell Cultures GmbH.

Clones from the transformation were selected on LBamp plates. LBamp contained 10 g/l tryptone, 5 g/l yeast extract, 5 g/l NaCl, 15 g/l agar and 100 mg/L ampicillin (Sigma-Aldrich). A clone was selected and cultivated in a shake flask growth. The CSma-producing strain was designated E. coli JM105×pCSma-pKKj. The CSma cds was expressed in E. coli JM105×pCSma-pKKj in a known manner under the control of the IPTG-inducible tac promoter (IPTG: isopropyl β-thiogalactoside, Sigma-Aldrich) functionally linked to the CSma cds.

### Growth in a Shake Flask:

A preculture of the strain E. coli JM105×pCSma-pKKj was prepared in LBamp medium (growth at 37° C. and 120 rpm overnight, Infors chest shaker).

Two ml of preculture (OD₆₀₀ 3.4/ml) were used as inoculum for a main culture (0.3 L Erlenmeyer flask) of 50 ml of SM1 medium (example 2), supplemented with 15 g/L glucose; 5 g/L peptone (Oxoid); 2.5 g/L yeast extract; 0.005 g/L vitamin B1 (Sigma-Aldrich); 5 mg/L pyridoxal phosphate (PLP, Sigma-Aldrich) and 100 mg/L ampicillin. The main culture was shaken at 30° C. and 140 rpm in a chest shaker (Infors). After a 4 h incubation time, a cell density OD₆₀₀ of 2.0 was reached. Then the inducer IPTG (Sigma-Aldrich, 0.4 mM final concentration) was added and growth was continued for another 20 h at 30° C. and 140 rpm in a chest shaker (Infors). At the end of growth, the cell density OD₆₀₀ was 3.1/ml.

The cells from the shake flask growth were isolated by centrifugation (10 min at 15 000 rpm, Sorvall RC5C centrifuge, equipped with an SS34 rotor). The cell pellet from 50 ml of shake flask growth was resuspended in 2 ml of 100 mM K phosphate, pH 7.0; 100 mM KCl (KPi7.0 buffer) to prepare a cell suspension and used for preparation of a cell homogenate. A cell homogenate was prepared using the FastPrep-24™ 5G cell homogenizer from MP Biomedicals, as described in example 2. The cell homogenate obtained (2 ml volume) was used without further workup for the biotransformation of OPS to L-cysteic acid (example 5).

The protein content of the cell homogenate was determined by means of a Qubit 3.0 Fluorometer from Thermo Fisher Scientific using the “Qubit© Protein Assay Kit” according to the manufacturer's instructions. The protein content of the cell homogenate from the shake flask growth was 5.3 mg/ml.

### Example 4: Production of L-Cysteic Acid from Commercially Available OPS and Na₂SO₃ by Biotransformation with CSma Enzyme

Two batches were carried out in parallel:
Batch 1: A 100 ml Erlenmeyer flask was initially charged with 8.15 ml of KPi7.0 buffer, and added in succession were 1 ml of a 0.2 M solution of Na₂SO₃ in KPi7.0 buffer buffer, 0.5 ml of CSma cell homogenate from the shake flask growth (example 3) and 350 μl of a 0.2 M solution of OPS (Sigma-Aldrich) in KPi7.0 buffer. The batch volume was 10 ml.
Batch 2: The batch (comparative batch without Na₂SO₃) had the same composition as batch 1. Instead of the Na₂SO₃ solution, batch 2 received 1 ml of KPi7.0 buffer buffer.

Both batches were incubated at 30° C. and 140 rpm in a chest shaker (Infors). After 1 h, 2 h and 4 h, 1 ml of the batches was in each case incubated at 80° C. for 5 min and centrifuged and the supernatant was analyzed by HPLC. The time course of the production of L-cysteic acid from OPS is shown in Table 3.

TABLE 3
Time course of the production of L-cysteic acid by biotransformation
of OPS and Na₂SO₃ with CSma-containing cell homogenate

| | Batch 1 with Na₂SO₃ | Batch 2 without Na₂SO₃ |
|---|---|---|
| | L-cysteic acid | L-cysteic acid |
| Time | concentration | concentration |
| [h] | [mg/L] | [mg/L] |
| 0 | 0.0 | 0.0 |
| 1 | 136.5 | 0.0 |
| 2 | 253.3 | 0.0 |
| 4 | 473.1 | 0.0 |

### Example 5: Production of L-Cysteic Acid from OPS-Containing Culture Supernatant from the Shake Flask Growth and Na₂SO₃ by Biotransformation with CSma Enzyme

A 100 ml Erlenmeyer flask was initially charged with 9 ml of cell culture supernatant from the shake flask growth of the strain E. coli W3110-ΔserB (example 2) having an OPS content of 113.6 mg/L, and added were 0.3 ml of 3 M KCl, 0.5 ml of 0.2 M Na₂SO₃ in KPi7.0 buffer and 1 ml of CSma cell homogenate from the shake flask growth (example 3). The batch volume was 10.8 ml. The batch was incubated at 30° C. and 140 rpm in a chest shaker (Infors). At the start and after 6 h, 1 ml of the batch was in each case incubated at 80° C. for 5 min and centrifuged and the supernatant was analyzed by HPLC for the content of OPS and L-cysteic acid. The course of the reaction over time is summarized in Table 4. The molar yield of L-cysteic acid, based on the molar amount of OPS used, was 97.6%.

TABLE 4
Time course of the production of L-cysteic acid from
OPS-containing cell culture supernatant and Na₂SO₃ by
catalysis with CSma-containing cell homogenate

| | OPS | OPS | L-Cysteic acid | L-Cysteic acid |
|---|---|---|---|---|
| Time | concentration | concentration | concentration | concentration |
| [h] | [mg/L] | [mM] | [mg/L] | [mM] |
| 0 | 94.7 | 0.51 | 0.00 | 0.00 |
| 6 | 0.00 | 0.00 | 84.5 | 0.50 |

### Example 6: Preparative Production of L-Cysteic Acid by Biotransformation of OPS at Constant pH

OPS substrate: 10 ml of fermenter broth from the fermentation of the strain E. coli W3110-ΔserB (example 2) were centrifuged (10 min 15 000 rpm, Sorvall RC5C centrifuge, equipped with an SS34 rotor) and the OPS content in the fermentation supernatant was determined by HPLC. The OPS content was 5.6 g/L.

CSma homogenate: Cell homogenate of the strain E. coli JM105×pCSma-pKKj was prepared from 2×50 ml of shake flask growth, as described in example 3. The homogenate was supplemented with 15 μl of 500 mg/L PLP. The total volume of the homogenate was 4 ml.

Biotransformation batch: A 50 ml thermostatable double-walled and downward tapering reaction vessel (accessory for Titrator TitroLine alpha, Schott) was connected to a thermostat (Lauda) via a hose connection and adjusted to a temperature of 30° C.

The reaction batch contained 6 ml of OPS-containing fermentation supernatant having an OPS content of 5.6 g/L, 4 ml of CSma homogenate, 0.2 ml of 3 M KCl, 0.1 ml of 0.1 M DTE (dithioerythrol, Sigma-Aldrich), and 0.3 ml of a 1 M solution of Na₂SO₃ in KPi7.0 buffer. The batch volume was 10.6 ml. The OPS concentration in the batch was 17.1 mM (0.18 mmol of OPS in a 10.6 ml batch volume). The batch was stirred with a magnetic stirrer. The batch was also equipped with a pH electrode (Mettler Toledo), which was connected to a pH control unit (TitroLine alpha titrator, Schott) which was operated in pH-stat mode according to the manufacturer's instructions. Under pH-stat conditions, the pH in the reaction vessel was kept constant at the set pH of 7.0 over the entire duration of the reaction by metered addition of 0.5 M NaOH from a burette connected to the control unit.

The reaction time was 4 h. Since the batch was supplied with 0.5 M NaOH from the burette to maintain a constant pH of 7.0, the batch volume was 13.0 ml after the 4 h reaction time. 2 h and 4 h after the start of the reaction, 50 μl aliquots of the batch were removed in each case and the content of OPS and L-cysteic acid was analyzed by HPLC. The course of the reaction over time is summarized in Table 5. After a 4 h reaction time, the L-cysteic acid content in the batch was 2399.6 mg/L (14.2 mM), which corresponded to an absolute molar yield of 0.18 mmol of L-cysteic acid for a batch volume of 13.0 ml. OPS used was completely consumed. Based on the amount of OPS used of 0.18 mmol, this corresponded to a yield of 100%.

TABLE 5
HPLC-detected amount of OPS and L-cysteic acid according
to reaction time, using an OPS-containing fermentation supernatant,
Na₂SO₃ and a CSma-containing cell homogenate from
the shake flask growth

| | | OPS | OPS | | |
|---|---|---|---|---|---|
| | Batch | concen- | concen- | L-Cysteic acid | L-Cysteic acid |
| Time | volume | tration | tration | concentration | concentration |
| [h] | [ml] | [mg/L] | [mM] | [mg/L] | [mM] |
| 0 | 10.6 | 3170.0 | 17.13 | 0.0 | 0.00 |
| 2 | 12.8 | 918.3 | 4.96 | 1648.6 | 9.7 |
| 4 | 13.0 | 0.0 | 0.00 | 2399.6 | 14.2 |

### Example 7: Recombinant Production of CSADcc from Cyprinus carpio (Carp) in E. Coli

### CSADcc Gene:

The mRNA-derived cDNA sequence of the cysteine sulfinic acid decarboxylase (CSAD) from Cyprinus carpio (carp) is disclosed in the NCBI database (National Center for Biotechnology Information) under the Genbank sequence ID: AB220585.1 (cds: nt 82-1584). The corresponding amino acid sequence was used to derive a DNA sequence codon-optimized for expression in E. coli (publicly available Eurofins Genomics GENEius software), which was synthetically produced (Eurofins Genomics). The synthetically produced DNA had the sequence disclosed in SEQ ID NO: 5 and contained the cds of the gene, referred to hereinafter as CSADcc cds (SEQ ID NO: 5, nt 31-1530), encoding a protein having the amino acid sequence disclosed in SEQ ID NO: 6 and referred to as CSADcc. For cloning purposes, the synthetically produced DNA contained at the 5′ end an EcoRI cleavage site (SEQ ID NO: 5, nt 25 to 30) and at the 3′ end a HindIII cleavage site (SEQ ID NO: 5, nt 1532 to 1537).

Vector pCSADcc-pKKj:

The vector pCSADcc-pKKj suitable for recombinant expression of the CSADcc cds (FIG. 4) was produced by cutting the synthetically produced DNA using EcoRI and HindIII and cloning it in a known manner as an EcoRI/HindIII fragment into the vector pKKj cut using EcoRI and HindIII (see example 3). This yielded the vector pCSADcc-pKKj. The CSADcc cds was expressed in E. coli by transforming the vector pCSADcc-pKKj in a known manner into the strain E. coli K12 JM105. Clones from the transformation were selected on LBamp plates. A clone was selected and cultivated in a shake flask growth. The CSADcc-producing strain was designated E. coli JM105×pCSADcc-pKKj. CSADcc cds was expressed in E. coli JM105×pCSADcc-pKKj analogously as described in example 3 for E. coli JM105×pCSma-pKKj by growth in a shake flask.

Growth in a shake flask: 10 ml of a preculture (OD₆₀₀ 3.1/ml) were used as inoculum for a main culture (1 L Erlenmeyer flask) of 100 ml of SM1 medium, supplemented with 15 g/L glucose; 5 g/L peptone; 2.5 g/L yeast extract; 0.005 g/L vitamin B1; 5 mg/L pyridoxal phosphate and 100 mg/L ampicillin. The main culture was grown and induced as described in example 3. The cells from 100 ml of the shake flask growth (OD₆₀₀ 7.1/ml) were isolated by centrifugation and the cell pellet was resuspended in 4 ml of KPi7.0 buffer. The cell suspension was used directly for biotransformation experiments.

### Example 8: Production of Taurine from L-Cysteic Acid by Biotransformation

A 100 ml Erlenmeyer flask was initially charged with 9 ml of the batch from example having a content of 84.5 mg/L L-cysteic acid (Table 4), and 1 ml of cell suspension of the CSADcc enzyme from example 7 was added. The batch volume was 10 ml. The batch was incubated at 37° C. and 140 rpm in a chest shaker (Infors). After 2 h, 1 ml of the batch was incubated at 80° C. for 5 min and centrifuged and the supernatant was analyzed by HPLC. The L-cysteic acid used had been completely consumed. The amount of taurine formed was 66.4 mg/L.

### Example 9: Production of Taurine from OPS by Biotransformation

A 100 ml Erlenmeyer flask was initially charged with 9 ml of a batch from the shake flask growth of the strain E. coli W3110-ΔserB (example 2) having a content of 94.7 mg/L OPS, and added were 0.5 ml of a 0.2 M solution of Na₂SO₃ in KPi7.0 buffer buffer, 1 ml of CSma cell homogenate from the shake flask growth (example 3) and 1 ml of cell suspension of the CSADcc enzyme (example 7). The batch volume was 11.5 ml. The batch was incubated at 30° C. and 140 rpm in a chest shaker (Infors). After 4 h, 1 ml of the batch was incubated at 80° C. for 5 min and centrifuged and the supernatant was analyzed by HPLC. The OPS used was completely consumed. At the same time, 34.4 mg/L L-cysteic acid and 40.4 mg/L taurine had been formed.

### Abbreviations Used in the Figures

bla: Gene conferring resistance to ampicillin (β-lactamase)
kanR: Gene conferring resistance to kanamycin
ORI: Origin of replication
pr-1: Binding site 1 for primer
pr-2: Binding site 2 for primer
FRT1: Recognition sequence 1 for FLP recombinase
FRT2: Recognition sequence 2 for FLP recombinase
araC: araC gene (repressor gene)
P araC: Promoter of the araC gene
P araB: Promoter of the araB gene
Gam: Lambda phage Gam recombination gene
Bet: Lambda phage Bet recombination gene
Exo: Lambda phage Exo recombination gene
ORI101: Temperature-sensitive origin of replication
RepA: Gene for plasmid replication protein A
Ptac: tac promoter
EcoRI: Cleavage site for the restriction enzyme EcoRI
HindIII: Cleavage site for the restriction enzyme HindIII
CSma: cds of the cysteate synthase gene from M. acetivora
CSADcc: cds of the cysteine sulfinic acid decarboxylase gene from C. carpio

1-14. (canceled)

15. A process for producing L-cysteic acid, comprising:
a reaction of O-phospho-L-serine (OPS) with a salt of sulfurous acid (sulfite) and a cysteate synthase (CS enzyme) belonging to enzyme class EC 2.5.1.76 in a biotransformation;
wherein a concentration of OPS in a batch is at least 1 g/L;
wherein the CS enzyme is produced in an enzymatically active form by growth of the microorganism strain E. coli K12 JM105;
wherein the biotransformation is carried out under active pH control and the CS enzyme has an amino acid sequence specified in SEQ ID NO: 4 or an amino acid sequence homologous thereto; and
wherein an amino acid sequence homologous to SEQ ID NO: 4 has a sequence identity of at least 50% in relation to SEQ ID NO: 4 and, at the same time, cysteate synthase enzyme activity.

16. The process of claim 15, wherein the CS enzyme is used in the reaction without a prior renaturation step.

17. The process of claim 15, wherein the OPS used in the reaction is produced biotechnologically.

18. The process of claim 15, wherein the OPS used in the reaction is produced using a microorganism strain having suppressed activity of the O-phospho-L-serine phosphatase (SerB enzyme) belonging to enzyme class EC 3.1.3.3.

19. The process of claim 15, wherein a molar yield of L-cysteic acid based on a molar amount of OPS used is at least 60%.

20. The process of claim 15, wherein the L-cysteic acid produced is used in the production of taurine.

21. A process for producing taurine, comprising:
providing L-cysteic acid, wherein the L-cysteic acid is produced by a reaction of O-phospho-L-serine (OPS) with a salt of sulfurous acid (sulfite) and a cysteate synthase (CS enzyme) belonging to enzyme class EC 2.5.1.76 in a biotransformation,
wherein a concentration of OPS in a batch is at least 1 g/L,
wherein the CS enzyme is produced in an enzymatically active form by growth of the microorganism strain E. coli K12 JM105,
wherein the biotransformation is carried out under active pH control and the CS enzyme has an amino acid sequence specified in SEQ ID NO: 4 or an amino acid sequence homologous thereto, and
wherein an amino acid sequence homologous to SEQ ID NO: 4 has a sequence identity of at least 50% in relation to SEQ ID NO: 4 and, at the same time, cysteate synthase enzyme activity; and
wherein the L-cysteic acid is decarboxylated.

22. The process of claim 21, wherein the decarboxylation is effected by means of a cysteine sulfinic acid decarboxylase (CSAD enzyme) belonging to enzyme class EC 4.1.1.29.

23. The process of claim 22, wherein the amino acid sequence of the CSAD enzyme is SEQ ID NO. 6.

24. The process of claim 21, wherein a molar yield of taurine based on a molar amount of L-cysteic acid used is at least 60%.

25. The process of claim 21, wherein all of the process steps take place in one reaction batch (one-pot process).


SEQUENCE LISTING (10 sequences)

SEQ ID NO: 1 - DNA, 1085 bp, Escherichia coli
  gene (1)..(1085)
  gene (1)..(1085): serB
tgagacttaatgttgccagaagcaatggttattttccctgcttcgaacgattttacaggagccttaatgcctaacattacctggtgcgacctgcctgaagatgtctctttatggccgggtctgcctctttcattaagtggtgatgaagtgatgccactggattaccacgcaggtcgtagcggctggctgctgtatggtcgtgggctggataaacaacgtctgacccaataccagagcaaactgggtgcggcgatggtgattgttgccgcctggtgcgtggaagattatcaggtgattcgtctggcaggttcactcaccgcacgggctacacgcctggcccacgaagcgcagctggatgtcgccccgctggggaaaatcccgcacctgcgcacgccgggtttgctggtgatggatatggactccaccgccatccagattgaatgtattgatgaaattgccaaactggccggaacgggcgagatggtggcggaagtaaccgaacgggcgatgcgcggcgaactcgattttaccgccagcctgcgcagccgtgtggcgacgctgaaaggcgctgacgccaatattctgcaacaggtgcgtgaaaatctgccgctgatgccaggcttaacgcaactggtgctcaagctggaaacgctgggctggaaagtggcgattgcctccggcggctttactttctttgctgaatacctgcgcgacaagctgcgcctgaccgccgtggtagccaatgaactggagatcatggacggtaaatttaccggcaatgtgatcggcgacatcgtagacgcgcagtacaaagcgaaaactctgactcgcctcgcgcaggagtatgaaatcccgctggcgcagaccgtggcgattggcgatggagccaatgacctgccgatgatcaaagcggcagggctggggattgcctaccatgccaagccaaaagtgaatgaaaaggcggaagtcaccatccgtcacgctgacctgatgggggtattctgcatcctctcaggcagcctgaatcagaagtaattgctcgcccgccatcctgcgggcggcacagcattaacgaggtacaccgt

SEQ ID NO: 2 - protein, 322 aa, Escherichia coli [sequence omitted]

SEQ ID NO: 3 - DNA, 1251 bp, Methanosarcina acetivorans
  gene (1)..(1251)
  gene (1)..(1251): CSma
atgggccgctttatcctgaaatgcctgaaatgtggccgtgaatatagtcaggagtatcgcttgacttgcgaaaacgacgatagcttcctgcgtgccgaatacctggaaaagaaacttgagctgcgtaaacaacctgggattggacgtttccattcgtggctgccggtacaggaagagctgaccacggaagcgggtcccattacgtacaaatcggaggcattagcccgcgaattagggctgtcaaacttgtacatcggctttagcggctattggccggagaaaggcgcctttatcaaaacctgtagcttcaaagagttagaagcgcatcctacaatgcagctgctgaaggaaagcggcgggaaggccattgtgcttgcttctgcaggcaacactggacgggcgtttgcgcatgtgagtgccctcaccggaaccgacgtgtacattgtggttccagactcaggcattccgaaactgtggttgccagaagaaccgaccgactctattcaccttatcagcatgaccccagggaacgactataccgatgccattaatctggccggacgcattgcgaaattaccgggtatggttcctgaaggcggtgcacgcaacgtggctcgtcgcgaaggcatgggcactgtgatgctggatgcagcggtaacgatcggtaaaatgccggatcactactttcaagccgttggcagtggcacaggcggtatttcagcctgggaagctagcttacgcctgcgtgaagatggtcgctttgggtccaaactgccgaaactccagcttactcagaacctcccgtttgtccccatgtataacgcgtggcaagagggtcgccgggatattatcccggaaattgacatgaaagatgcgaagaagcgtatcgaggaaacgtatgcaaccgtcctgaccaatcgtgcaccgccgtattccgtgacaggtggtctgtacgatgcgttggtcgataccgatggcatcatgtatgcggtttccaaagaagaagccttggatgcgaaagcgctgttcgaaagtctcgaaggtatcgatattctgccgccatctgctgttgctgcagcttcgttgctcaaagcagtcgaagcgggtaatgtcgggaaagacgacacgattctgctgaatatcgcgggtggcggtttcaaacgccttaaggaagatttcaccctgtttcagattgagccggagattacggtgtcgaatcccgatgtacctctggaagagctgaaattataa

SEQ ID NO: 4 - protein, 416 aa, Methanosarcina acetivorans
  PEPTIDE (1)..(416)
  PEPTIDE (1)..(416): CSma Protein
MGRFILKCLKCGREYSQEYRLTCENDDSFLRAEYLEKKLELRKQPGIGRFHSWLPVQEELTTEAGPITYKSEALARELGLSNLYIGFSGYWPEKGAFIKTCSFKELEAHPTMQLLKESGGKAIVLASAGNTGRAFAHVSALTGTDVYIVVPDSGIPKLWLPEEPTDSIHLISMTPGNDYTDAINLAGRIAKLPGMVPEGGARNVARREGMGTVMLDAAVTIGKMPDHYFQAVGSGTGGISAWEASLRLREDGRFGSKLPKLQLTQNLPFVPMYNAWQEGRRDIIPEIDMKDAKKRIEETYATVLTNRAPPYSVTGGLYDALVDTDGIMYAVSKEEALDAKALFESLEGIDILPPSAVAAASLLKAVEAGNVGKDDTILLNIAGGGFKRLKEDFTLFQIEPEITVSNPDVPLEELKL

SEQ ID NO: 5 - DNA, 1566 bp, Cyprinus carpio
  gene (1)..(1566)
  gene (1)..(1566): CSADcc
gttaacaatttcacacaggaaacagaattcatgagttcgtccaaggaatatatgaatggccacgtgcatctggaggagagtgatatgtatgaatccgatggcaaactgtttctgacggaagcctttaacgttatcatggaagaaattttgcacaaaggtaccgattcggaagagaaagtgtgtgagtggaaagatcccgatcagctgcgtgccctcttggatctggatctgcgtgaacatggggaatcacataaacagctgcttcaacgcgtgcgtgacgttgcacgctattctgtcaaaacctgtcatccgcgcttcttcaatcagctgtttgccggcgtagactatcacgctcttaccggacgcttgctgaccgaaaccctgaatacgagtcagtacacttacgaagtcgctccggtgtttgttcttatggaagaggaagtgctttgcaaattacgcagcctcgtcggatggtctgaaggcgacggtattttctgtcctggcggcagcatgtccaacatgtatgcgatgaatgtggctcgttattgggctttccctcaggttaaaacgcagggtttatgggcaacgccacgtatggcgattttcacctctcaacagggccattacagcatgaagaaagcggcagcctttctgggtattggcacagacaatgtgttcatcgtacaggtcgatgaaagcgggagtatgcgtcctgaggatctcgaagcgaaaatcgtacaagcgaaaagccaggatgccgttcccttctttgtgaacgcaacagccggtacaaccgttcaaggtgcctttgatccgctgaatcgtattgcggacatctcagaacgcaacggcatgtggatgcacgtcgatgcggcttggggtggttcagtgctgtttagcaagaagcacaaacatctggttgcgggcattgaacgtgcaaacagcgtgacttggaatccgcacaaaatgctgctggccgggttacagtgttctgtcgtactgtttcgcgataccacgaatttactgatgcactgccattcggccaaagcgacgtacctcttccaacaagacaagttttacgacacttcgcttgatactggggacaaaagcattcagtgcggtcgcaaggtggattgcttgaaactgtggctgatgtggaaagcgattggcacccatgggttatccgagcgtgtagagaaagcgtttgctttagcacgctatctggtcgaagaaatggaaaagcgcgacaacttcaagctggtgtgcaaaggtccgtttgtaaacgtgtgcttttggtttatcccgccgtcactccgccgcaaagaaaactccgcggattaccaagaacggttgtcgaaagtggcaccggttatcaaagagcggatgatgaaacgtggaacgatgatggtgggttatcagccaatggatggccatgtcaacttctttcgcatggttgttgtcagtccacagttgaccaccaaagacatggacttcttcctggatgagattgaagatctgggcaaagacctgtaagcttggctgttttggcggatgagagaagatatc

SEQ ID NO: 6 - protein, 500 aa, Cyprinus carpio
  PEPTIDE (1)..(500)
  PEPTIDE (1)..(500): CSADcc Protein
MSSSKEYMNGHVHLEESDMYESDGKLFLTEAFNVIMEEILHKGTDSEEKVCEWKDPDQLRALLDLDLREHGESHKQLLQRVRDVARYSVKTCHPRFFNQLFAGVDYHALTGRLLTETLNTSQYTYEVAPVFVLMEEEVLCKLRSLVGWSEGDGIFCPGGSMSNMYAMNVARYWAFPQVKTQGLWATPRMAIFTSQQGHYSMKKAAAFLGIGTDNVFIVQVDESGSMRPEDLEAKIVQAKSQDAVPFFVNATAGTTVQGAFDPLNRIADISERNGMWMHVDAAWGGSVLFSKKHKHLVAGIERANSVTWNPHKMLLAGLQCSVVLFRDTTNLLMHCHSAKATYLFQQDKFYDTSLDTGDKSIQCGRKVDCLKLWLMWKAIGTHGLSERVEKAFALARYLVEEMEKRDNFKLVCKGPFVNVCFWFIPPSLRRKENSADYQERLSKVAPVIKERMMKRGTMMVGYQPMDGHVNFFRMVVVSPQLTTKDMDFFLDEIEDLGKDL

SEQ ID NO: 7 - DNA, 50 bp, Artificial Sequence
  Primer
  misc_feature (1)..(50)
  misc_feature (1)..(50): serb-1f
atgcctaacattacctggtgcgacctgcctgtgtaggctggagctgcttc

SEQ ID NO: 8 - DNA, 50 bp, Artificial Sequence
  Primer
  misc_feature (1)..(50)
  misc_feature (1)..(50): serb-2r
ttacttctgattcaggctgcctgagaggatctgtcaaacatgagaattaa

SEQ ID NO: 9 - DNA, 22 bp, Artificial Sequence
  Primer
  misc_feature (1)..(22)
  misc_feature (1)..(22): serb-3f
tgagacttaatgttgccagaag

SEQ ID NO: 10 - DNA, 20 bp, Artificial Sequence
  Primer
  misc_feature (1)..(20)
  misc_feature (1)..(20): serb-4r
acggtgtacctcgttaatgc